(19) United States
(12) Patent Application Publication
(10) Pub. No.: US 2024/0188798 A1
(43) Pub. Date: Jun. 13, 2024

(54) ENDOSCOPE SYSTEM, MEDICAL INFORMATION PROCESSING APPARATUS, MEDICAL INFORMATION PROCESSING METHOD, MEDICAL INFORMATION PROCESSING PROGRAM, AND RECORDING MEDIUM

(71) Applicant: FUJIFILM Corporation, Tokyo (JP)

(72) Inventors: Yuya KIMURA, Kanagawa (JP); Yuma HORI, Kanagawa (JP); Tatsuya KOBAYASHI, Kanagawa (JP); Shigetoshi ISHIKAWA, Kanagawa (JP); Eiichi IMAMICHI, Kanagawa (JP)

(73) Assignee: FUJIFILM Corporation, Tokyo (JP)

(21) Appl. No.: 18/582,652

(22) Filed: Feb. 21, 2024

Related U.S. Application Data
(63) Continuation of application No. PCT/JP2022/033261, filed on Sep. 5, 2022.

(30) Foreign Application Priority Data
Sep. 8, 2021 (JP) 2021-146309

(51) Int. Cl. A61B 1/00

(52) U.S. Cl. CPC A61B 1/000096; A61B 1/0002; A61B 1/0004; A61B 1/0005

(57) ABSTRACT

According to the present disclosure, it is possible to smoothly proceed with an examination in which an audio input and audio recognition are performed on medical images. In the endoscope system, a processor acquires a plurality of medical images by causing an image sensor to image a subject in chronological order, accepts an input of an audio input trigger during capturing of the plurality of medical images, sets, in a case where the audio input trigger is input, an audio recognition dictionary according to the audio input trigger, performs, in a case where the audio recognition dictionary is set, audio recognition on audio input to the audio input device after the setting, using the set audio recognition dictionary, and displays item information indicating an item to be recognized using the audio recognition dictionary, and a result of audio recognition corresponding to the item information, on a display device.

### CROSS-REFERENCE TO RELATED APPLICATIONS

The present application is a Continuation of PCT International Application No. PCT/JP2022/033261 filed on Sep. 5, 2022 claiming priority under 35 U.S.C § 119(a) to Japanese Patent Application No. 2021-146309 filed on Sep. 8, 2021. Each of the above applications is hereby expressly incorporated by reference, in its entirety, into the present application.

### BACKGROUND OF THE INVENTION

### 1. Field of the Invention

The present invention relates to an endoscope system, a medical information processing apparatus, a medical information processing method, a medical information processing program, and a recording medium which perform an audio input and audio recognition.

### 2. Description of the Related Art

In the technical field of performing an examination and diagnosis support using medical images, it is known to recognize an audio input by a user and to perform processing based on a recognition result. In addition, it is also known to display information input by audio. For example, JP2013-106752A and JP2006-221583A disclose that input audio information is displayed in chronological order.

### SUMMARY OF THE INVENTION

In a case of performing an audio input during an examination using medical images, in a case where all words can be recognized regardless of the scene, there is a risk that mutual erroneous recognition between words increases and operability is reduced. In addition, since a display device displays various kinds of information during the examination, depending on a display mode, necessary information may not be displayed appropriately, which may hinder the examination (examination procedure). However, the techniques in the related art such as JP2013-106752A and JP2006-221583A described above have not sufficiently taken these problems into consideration.

The present invention has been made in view of such circumstances, and an object thereof is to provide an endoscope system, a medical information processing apparatus, a medical information processing method, a medical information processing program, and a recording medium capable of smoothly proceeding with an examination in which an audio input and audio recognition are performed on medical images.

In order to achieve the object described above, an endoscope system according to a first aspect of the present invention is an endoscope system including an audio input device; an image sensor that images a subject; and a processor, in which the processor acquires a plurality of medical images obtained by the image sensor imaging the subject in chronological order, accepts an input of an audio input trigger during capturing of the plurality of medical images, sets, in a case where the audio input trigger is input, an audio recognition dictionary according to the audio input trigger, performs, in a case where the audio recognition dictionary is set, audio recognition on audio input to the audio input device after the setting, using the set audio recognition dictionary, and displays item information indicating an item to be recognized using the audio recognition dictionary, and a result of audio recognition corresponding to the item information, on a display device.

According to the first aspect, since an appropriate audio recognition dictionary is set according to the audio input trigger, it is possible to improve the accuracy of the audio recognition, and since the item information indicating the item to be recognized using the audio recognition dictionary and the result of the audio recognition corresponding to the item information are displayed on the display device, it is possible for the user to easily visually recognize the recognition result. Thereby, an examination in which the audio input and the audio recognition are performed on the medical image can proceed smoothly. Note that, in the first aspect, it is preferable that the processor displays the item information and the result of the audio recognition in association with each other.

In the endoscope system according to a second aspect, in the first aspect, in the audio recognition, the processor recognizes only registered words that are registered in the set audio recognition dictionary, and displays the result of the audio recognition for the registered words on the display device. According to the second aspect, only the registered words registered in the set audio recognition dictionary are audio-recognized, so that recognition accuracy can be improved.

In the endoscope system according to a third aspect, in the first aspect, in the audio recognition, the processor recognizes registered words that are registered in the set audio recognition dictionary and specific words, and displays the result of the audio recognition for the registered words among the recognized words on the display device. Note that an example of the “specific word” is a wake word for the audio input device, but the “specific word” is not limited thereto.

In the endoscope system according to a fourth aspect, in any one of first to third aspects, after the item information is displayed, the processor displays the result of the audio recognition corresponding to the displayed item information.

In the endoscope system according to a fifth aspect, in any one of the first to fourth aspects, in a case where any one of an imaging start instruction of the plurality of medical images, an output of a result of image recognition for the plurality of medical images, an operation to an operation device connected to the endoscope system, or an input of a wake word for the audio input device is performed, the processor decides that the audio input trigger is input.

In the endoscope system according to a sixth aspect, in any one of the first to fifth aspects, the processor determines whether or not a specific subject is included in the plurality of medical images, using image recognition, and accepts a determination result indicating that the specific subject is included, as the audio input trigger.

In the endoscope system according to a seventh aspect, in any one of the first to sixth aspects, the processor determines whether or not a specific subject is included in the plurality of medical images, using image recognition, discriminates the specific subject in a case where it is determined that the specific subject is included, and accepts an output of a discrimination result for the specific subject, as the audio input trigger.

In the endoscope system according to an eighth aspect, in any one of the first to seventh aspects, the processor performs image recognition a plurality of times, each with a different subject as a recognition target, on the plurality of medical images, and displays the item information corresponding to each of the plurality of times of image recognition and the result of the audio recognition.

In the endoscope system according to a ninth aspect, in the eighth aspect, the processor performs the image recognition a plurality of times using an image recognizer generated by machine learning.

In the endoscope system according to a tenth aspect, in any one of the first to ninth aspects, the processor displays information indicating that the audio recognition dictionary is set, on the display device.

In the endoscope system according to an eleventh aspect, in any one of the first to tenth aspects, the processor displays type information indicating a type of the set audio recognition dictionary, on the display device.

In the endoscope system according to a twelfth aspect, in any one of the first to eleventh aspects, the item information includes at least one of diagnosis, findings, treatments, or hemostasis.

In the endoscope system according to a thirteenth aspect, in any one of the first to twelfth aspects, the processor displays the item information and the result of the audio recognition on a same display screen as the plurality of medical images.

In the endoscope system according to a fourteenth aspect, in any one of the first to thirteenth aspects, the processor accepts confirmation information indicating confirmation of the audio recognition for one subject, ends, in a case where the confirmation information is accepted, display of the result of the audio recognition and the item information for the one subject, and accepts an input of the audio input trigger for another subject.

In the endoscope system according to a fifteenth aspect, in any one of the first to fourteenth aspects, the processor displays the item information and the result of the audio recognition during a display period after the setting, and ends the display in a case where the display period has elapsed.

In the endoscope system according to a sixteenth aspect, in the fifteenth aspect, the processor displays the item information and the result of the audio recognition during a period in which the audio recognition dictionary is set, as the display period, and ends the display of the item information and the result of the audio recognition in a case where the display period ends.

In the endoscope system according to a seventeenth aspect, in the fifteenth or sixteenth aspect, the processor displays the item information and the result of the audio recognition during a period with a length according to a type of the audio input trigger, as the display period, and ends the display of the item information and the result of the audio recognition in a case where the display period ends.

In the endoscope system according to an eighteenth aspect, in any one of the fifteenth to seventeenth aspects, the processor ends the display of the item information and the result of the audio recognition in a case where a state in which a specific subject is recognized in the plurality of medical images ends.

In the endoscope system according to a nineteenth aspect, in any one of the fifteenth to eighteenth aspects, the processor displays a remaining time of the display period on a screen of the display device.

In the endoscope system according to a twentieth aspect, in any one of the first to nineteenth aspects, the processor displays a candidate for recognition in the audio recognition on the display device, and confirms the result of the audio recognition on the basis of a selection operation of a user according to the display of the candidate.

In the endoscope system according to a twenty-first aspect, in the twentieth aspect, the processor accepts the selection operation via an operation device different from the audio input device.

In the endoscope system according to a twenty-second aspect, in any one of the first to twenty-first aspects, the processor records the plurality of medical images, the item information, and the result of the audio recognition in a recording device such that the plurality of medical images are associated with the item information, and the result of the audio recognition.

In order to achieve the object described above, a medical information processing apparatus according to a twenty-third aspect of the present invention is a medical information processing apparatus including a processor, in which the processor acquires a plurality of medical images obtained by an image sensor imaging a subject in chronological order, accepts an input of an audio input trigger during capturing of the plurality of medical images, sets, in a case where the audio input trigger is input, an audio recognition dictionary according to the audio input trigger, performs, in a case where the audio recognition dictionary is set, audio recognition on audio input to the audio input device after the setting, using the set audio recognition dictionary, and displays item information indicating an item to be recognized using the audio recognition dictionary, and a result of audio recognition corresponding to the item information, on a display device. According to the twenty-third aspect, similar to the first aspect, an examination in which the audio input and the audio recognition are performed on the medical image can proceed smoothly. Note that, in the twenty-third aspect, it is preferable that the processor displays the item information and the result of the audio recognition in association with each other. In addition, the twenty-third aspect may have the same configuration as the second to twenty-second aspects.

In order to achieve the object described above, a medical information processing method according to a twenty-fourth aspect of the present invention is a medical information processing method executed by an endoscope system including an audio input device, an image sensor that images a subject, and a processor, the medical information processing method including, via the processor, acquiring a plurality of medical images obtained by the image sensor imaging the subject in chronological order; accepting an input of an audio input trigger during capturing of the plurality of medical images; setting, in a case where the audio input trigger is input, an audio recognition dictionary according to the audio input trigger; performing, in a case where the audio recognition dictionary is set, audio recognition on audio input to the audio input device after the setting, using the set audio recognition dictionary; and displaying item information indicating an item to be recognized using the audio recognition dictionary, and a result of audio recognition corresponding to the item information, on a display device. According to the twenty-fourth aspect, similar to the first and twenty-third aspects, an examination in which the audio input and the audio recognition are performed on the medical image can proceed smoothly.

Note that, in the twenty-fourth aspect, it is preferable that the processor displays the item information and the result of the audio recognition in association with each other. In addition, the twenty-fourth aspect may have the same configuration as the second to twenty-second aspects.

In order to achieve the object described above, a medical information processing program according to a twenty-fifth aspect of the present invention is a medical information processing program causing an endoscope system including an audio input device, an image sensor that images a subject, and a processor to execute a medical information processing method, the medical information processing program causing, in the medical information processing method, the processor to acquire a plurality of medical images obtained by the image sensor imaging the subject in chronological order, accept an input of an audio input trigger during capturing of the plurality of medical images, set, in a case where the audio input trigger is input, an audio recognition dictionary according to the audio input trigger, perform, in a case where the audio recognition dictionary is set, audio recognition on audio input to the audio input device after the setting, using the set audio recognition dictionary, and display item information indicating an item to be recognized using the audio recognition dictionary, and a result of audio recognition corresponding to the item information, on a display device. According to the twenty-fifth aspect, similar to the first, twenty-third, and twenty-fourth aspects, an examination in which the audio input and the audio recognition are performed on the medical image can proceed smoothly.

Note that, in the twenty-fifth aspect, it is preferable that the processor displays the item information and the result of the audio recognition in association with each other. In addition, the medical information processing method that the medical information processing program according to the twenty-fifth aspect causes the endoscope system to execute may have the same configuration as the second to twenty-second aspects.

In order to achieve the object described above, a recording medium according to a twenty-sixth aspect of the present invention is a non-transitory and tangible recording medium in which a computer-readable code of the medical information processing program according to the twenty-fifth aspect is recorded. In the twenty-sixth aspect, examples of the “non-transitory and tangible recording medium” include various magneto-optical recording devices and semiconductor memories. The “non-transitory and tangible recording medium” does not include a non-tangible recording medium such as a carrier wave signal itself and a propagation signal itself.

Note that, in the twenty-sixth aspect, the medical information processing program of which the code is recorded in the recording medium may be one that causes the endoscope system or the medical information processing apparatus to execute a medical information processing program that performs the same processing as in the second to twenty-second aspects.

With the endoscope system, the medical information processing apparatus, the medical information processing method, the medical information processing program, and the recording medium according to the present invention, an examination in which the audio input and the audio recognition are performed on the medical image can proceed smoothly.

### BRIEF DESCRIPTION OF THE DRAWINGS

FIG. 1 is a diagram illustrating a schematic configuration of an endoscopic image diagnosis support system according to a first embodiment.

FIG. 2 is a diagram illustrating a schematic configuration of an endoscope system.

FIG. 3 is a diagram illustrating a schematic configuration of an endoscope.

FIG. 4 is a diagram illustrating an example of a configuration of an edge surface of a distal end portion.

FIG. 5 is a block diagram illustrating main functions of an endoscopic image generation device.

FIG. 6 is a block diagram illustrating main functions of an endoscopic image processing device.

FIG. 7 is a block diagram illustrating main functions of an image recognition processing unit.

FIG. 8 is a diagram illustrating an example of a screen display during an examination.

FIG. 9 is a diagram illustrating an outline of audio recognition.

FIGS. 10A to 10E are diagrams illustrating settings of an audio recognition dictionary.

FIGS. 11A and 11B are other diagrams illustrating settings of the audio recognition dictionary.

FIG. 12 is a time chart of setting the audio recognition dictionary.

FIGS. 13A to 13D are diagrams illustrating states of notification using a screen display of an icon.

FIGS. 14A to 14C are diagrams illustrating states of displaying a lesion information input box.

FIG. 15 is a diagram illustrating a basic display operation of the lesion information input box.

FIG. 16 is a time chart illustrating a display mode (Aspect 1) of the lesion information input box.

FIG. 17 is a diagram illustrating a state of a site selection in Aspect 1.

FIGS. 18A to 18C are diagrams illustrating states where information is input into the lesion information input box in Aspect 1.

FIG. 19 is a time chart illustrating a display mode (a modification example of Aspect 1) of the lesion information input box.

FIGS. 20A to 20C are diagrams illustrating states where information is input into the lesion information input box in the modification example.

FIG. 21 is a time chart illustrating a display mode (Aspect 2) of the lesion information input box.

FIGS. 22A to 22C are diagrams illustrating states where information is input into the lesion information input box in Aspect 2.

FIG. 23 is a time chart illustrating a display mode (Aspect 3) of the lesion information input box.

FIGS. 24A to 24D are diagrams illustrating states where information is input into the lesion information input box in Aspect 3.

FIGS. 25A and 25B are diagrams illustrating another display mode of the lesion information input box.

FIGS. 26A to 26C are diagrams illustrating still another display mode of the lesion information input box.

FIGS. 27A and 27B are diagrams illustrating still another display mode of the lesion information input box.

FIGS. 28A and 28B are diagrams illustrating still another display mode of the lesion information input box.

FIGS. 29A and 29B are diagrams illustrating variations in a findings input.

FIGS. 30A to 30C are diagrams illustrating variations in a findings input.

FIGS. 31A and 31B are diagrams illustrating examples of a screen display of a remaining time of an audio recognition period.

FIG. 32 is a diagram illustrating a state of executing an audio input during a specific period.

FIG. 33 is another diagram illustrating a state of executing an audio input during a specific period.

FIG. 34 is a diagram illustrating a state of processing according to a quality of image recognition.

### DESCRIPTION OF THE PREFERRED EMBODIMENTS

Embodiments of an endoscope system, a medical information processing apparatus, a medical information processing method, a medical information processing program, and a recording medium according to the present invention will be described. In the description, reference is made to the accompanying drawings as necessary. Note that, in the accompanying drawings, some constituents may be omitted for convenience of description.

### First Embodiment

### Endoscopic Image Diagnosis Support System

Here, a case where the present invention is applied to an endoscopic image diagnosis support system will be described as an example. The endoscopic image diagnosis support system is a system that supports detection and discrimination of a lesion or the like in an endoscopy. In the following, an example of application to an endoscopic image diagnosis support system that supports detection and discrimination of a lesion and the like in a lower digestive tract endoscopy (large intestine examination) will be described.

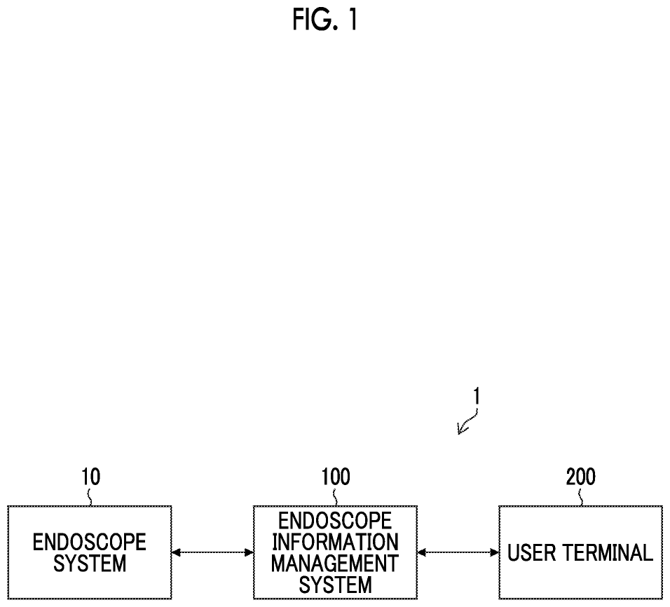

FIG. 1 is a block diagram illustrating an example of a schematic configuration of the endoscopic image diagnosis support system.

As illustrated in FIG. 1, an endoscopic image diagnosis support system 1 (endoscope system) of the present embodiment has an endoscope system 10 (endoscope system, medical information processing apparatus), an endoscope information management system 100, and a user terminal 200.

### Endoscope System

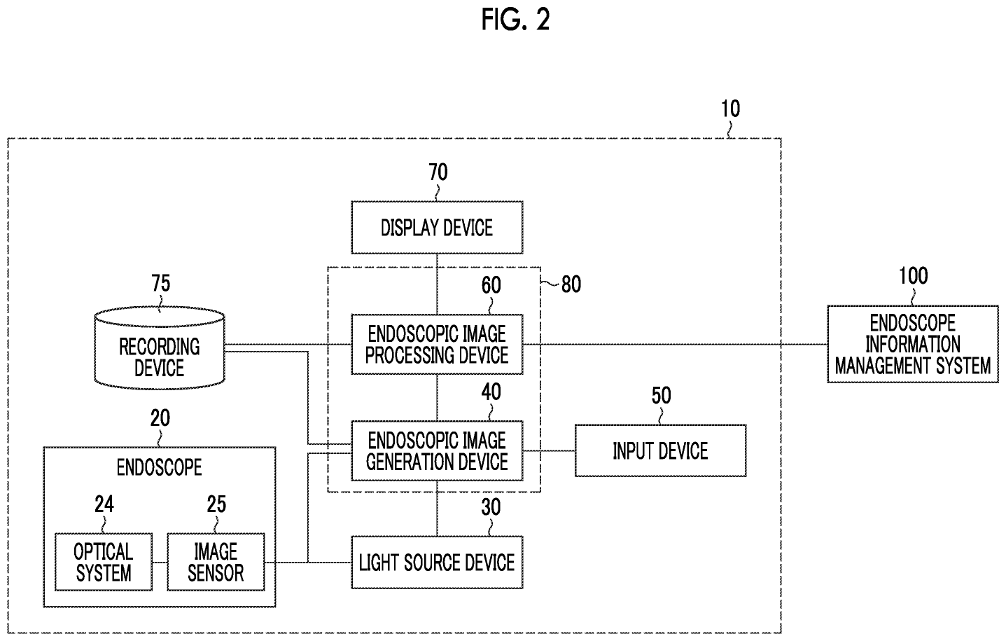

FIG. 2 is a block diagram illustrating a schematic configuration of the endoscope system 10.

The endoscope system 10 of the present embodiment is configured as a system capable of an observation using special light (special light observation) in addition to an observation using white light (white light observation). In the special light observation, a narrow-band light observation is included. In the narrow-band light observation, a blue laser imaging observation (BLI observation), a narrow band imaging observation (NBI observation; NBI is a registered trademark), a linked color imaging observation (LCI observation), and the like are included. Note that the special light observation itself is a well-known technique, so detailed description thereof will be omitted.

As illustrated in FIG. 2, the endoscope system 10 of the present embodiment has an endoscope 20, a light source device 30, an endoscopic image generation device 40, an endoscopic image processing device 60, a display device 70 (output device, display device), a recording device 75 (recording device), an input device 50, and the like. The endoscope 20 includes an optical system 24 built in a distal end portion 21A of an insertion part 21, and an image sensor 25 (image sensor). Note that the endoscopic image generation device 40 and the endoscopic image processing device 60 constitute a medical information processing apparatus 80 (medical information processing apparatus)

### Endoscope

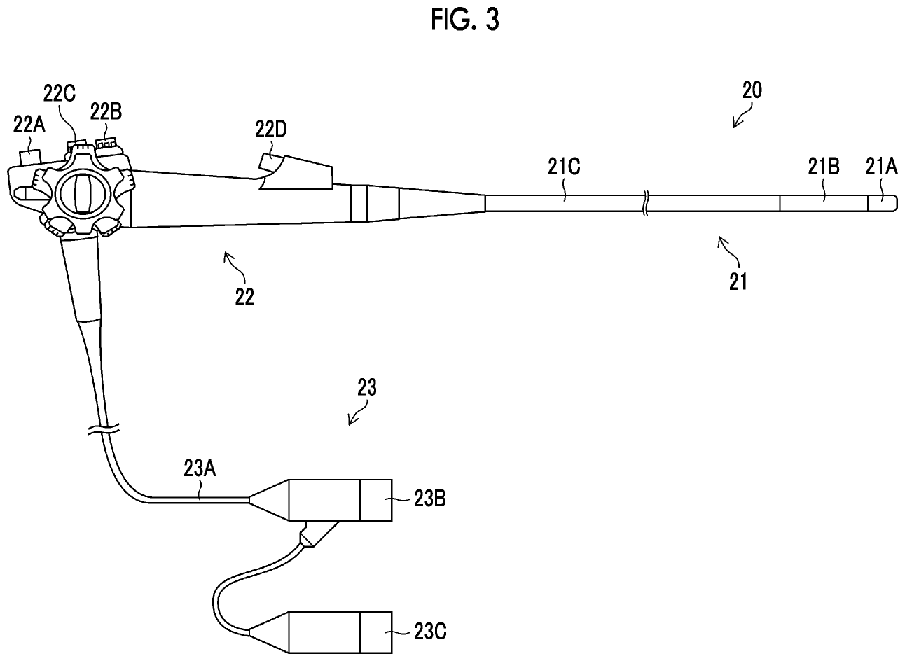

FIG. 3 is a diagram illustrating a schematic configuration of the endoscope 20.

The endoscope 20 of the present embodiment is an endoscope for a lower digestive organ. As illustrated in FIG. 3, the endoscope 20 is a flexible endoscope (electronic endoscope), and has the insertion part 21, an operation part 22, and a connection part 23.

The insertion part 21 is a part to be inserted into a hollow organ (large intestine in the present embodiment). The insertion part 21 includes the distal end portion 21A, a bendable portion 21B, and a soft portion 21C in order from a distal end side.

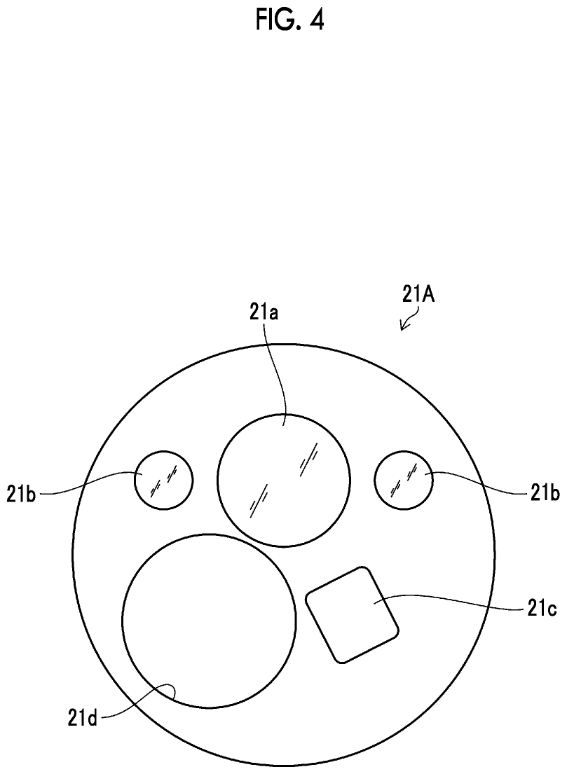

FIG. 4 is a diagram illustrating an example of a configuration of an edge surface of the distal end portion.

As illustrated in the figure, in the edge surface of the distal end portion 21A, an observation window 21a, illumination windows 21b, an air/water supply nozzle 21c, a forceps outlet 21d, and the like are provided. The observation window 21a is a window for an observation. The inside of the hollow organ is imaged through the observation window 21a. Imaging is performed via the optical system 24 such as a lens and the image sensor 25 (image sensor, refer to FIG. 2) built in the distal end portion 21A (portion of the observation window 21a). As the image sensor, for example, a complementary metal-oxide-semiconductor image sensor (CMOS image sensor), a charge-coupled device image sensor (CCD image sensor), or the like is used. The illumination windows 21b are windows for illumination. The inside of the hollow organ is irradiated with illumination light via the illumination windows 21b. The air/water supply nozzle 21c is a nozzle for cleaning. A cleaning liquid and a drying gas are sprayed from the air/water supply nozzle 21c toward the observation window 21a. The forceps outlet 21d is an outlet for a treatment tool such as forceps. The forceps outlet 21d functions as a suction port for sucking body fluids and the like.

The bendable portion 21B is a portion that is bent according to an operation of an angle knob 22A of the operation part 22. The bendable portion 21B is bent in four directions of up, down, left, and right.

The soft portion 21C is an elongated portion provided between the bendable portion 21B and the operation part 22. The soft portion 21C has flexibility.

The operation part 22 is a part that is held by an operator to perform various operations. The operation part 22 includes various operation members. As an example, the operation part 22 includes the angle knob 22A for a bending operation of the bendable portion 21B, an air/water supply button 22B for performing an air/water supply operation, and a suction button 22C for performing a suction operation. In addition, the operation part 22 includes an operation member (shutter button) for capturing a static image, an operation member for switching an observation mode, an operation member for switching on and off of various support functions, and the like. In addition, the operation part 22 includes a forceps insertion port 22D for inserting a treatment tool such as forceps. The treatment tool inserted from the forceps insertion port 22D is drawn out from the forceps outlet 21d (refer to FIG. 4) on a distal end of the insertion part 21. As an example, the treatment tool includes biopsy forceps, snares, and the like.

The connection part 23 is a part for connecting the endoscope 20 to the light source device 30, the endoscopic image generation device 40, and the like. The connection part 23 includes a cord 23A extending from the operation part 22, and a light guide connector 23B and a video connector 23C that are provided on a distal end of the cord 23A. The light guide connector 23B is a connector for connecting to the light source device 30. The video connector 23C is a connector for connecting to the endoscopic image generation device 40.

### Light Source Device

The light source device 30 generates illumination light. As described above, the endoscope system 10 of the present embodiment is configured as a system capable of the special light observation in addition to the normal white light observation. Therefore, the light source device 30 is configured to be capable of generating light (for example, narrow-band light) corresponding to the special light observation in addition to the normal white light. Note that, as described above, the special light observation itself is a well-known technique, so the description for the light generation will be omitted.

### Medical Information Processing Apparatus

### Endoscopic Image Generation Device

The endoscopic image generation device 40 (processor) comprehensively controls the entire operation of the endoscope system 10 together with the endoscopic image processing device 60 (processor). The endoscopic image generation device 40 includes, as a hardware configuration, a processor, a main storage unit (memory), an auxiliary storage unit (memory), a communication unit, and the like. That is, the endoscopic image generation device 40 has a so-called computer configuration as the hardware configuration. The processor is configured by, for example, a central processing unit (CPU), a graphics processing unit (GPU), a field-programmable gate array (FPGA), a programmable logic device (PLD), and the like. For example, the main storage unit is configured by a random-access memory (RAM) and the like. The auxiliary storage unit is configured by, for example, a non-transitory and tangible recording medium such as a flash memory, and can record computer-readable codes of a medical information processing program according to the embodiment of the present invention or of a part thereof, and other data. In addition, the auxiliary storage unit may include various magneto-optical recording devices, semiconductor memories, and the like in addition to or instead of the flash memory.

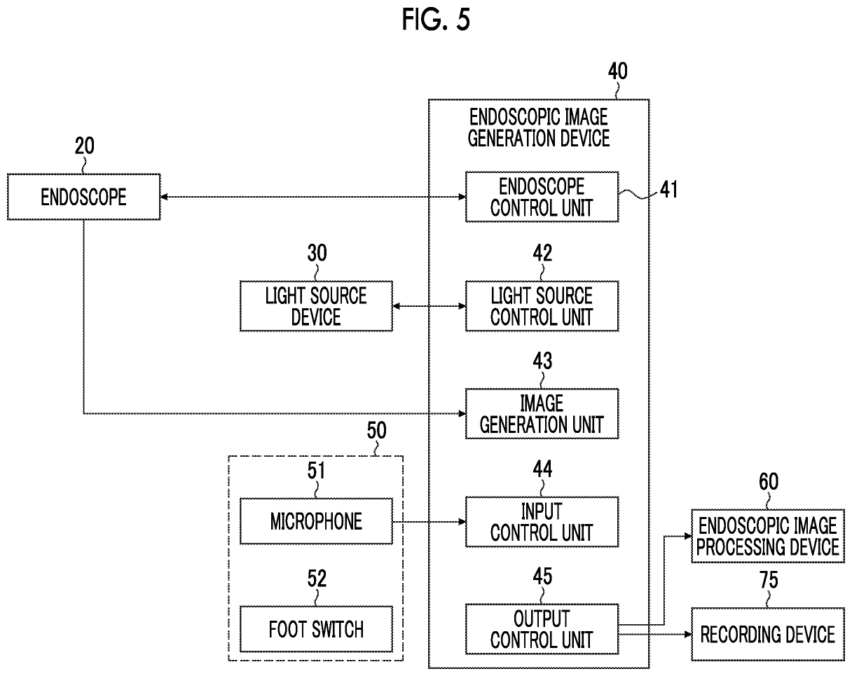

FIG. 5 is a block diagram illustrating main functions of the endoscopic image generation device 40.

As illustrated in the figure, the endoscopic image generation device 40 has functions of an endoscope control unit 41, a light source control unit 42, an image generation unit 43, an input control unit 44, an output control unit 45, and the like. Various programs executed by the processor (which may include the medical information processing program according to the embodiment of the present invention or a part thereof) and various kinds of data necessary for control or the like are stored in the auxiliary storage unit described above, and each function of the endoscopic image generation device 40 is realized by the processor executing these programs. The processor of the endoscopic image generation device 40 is an example of the processor in the endoscope system and in the medical information processing apparatus according to the embodiment of the present invention.

The endoscope control unit 41 controls the endoscope 20. The control for the endoscope 20 includes image sensor drive control, air/water supply control, suction control, and the like.

The light source control unit 42 controls the light source device 30. The control for the light source device 30 includes light emission control for a light source, and the like.

The image generation unit 43 generates captured images (endoscopic images) on the basis of signals output from the image sensor 25 of the endoscope 20. The image generation unit 43 can generate a static image and/or a video (a plurality of medical images obtained by the image sensor 25 imaging a subject in chronological order) as the captured image. The image generation unit 43 may perform various kinds of image processing on the generated images.

The input control unit 44 accepts an input of an operation and an input of various kinds of information via the input device 50.

The output control unit 45 controls an output of information to the endoscopic image processing device 60. The information to be output to the endoscopic image processing device 60 includes various kinds of operation information input from the input device 50, and the like in addition to the endoscopic image obtained by imaging.

### Input Device

The input device 50 constitutes a user interface in the endoscope system 10 together with the display device 70. The input device 50 includes a microphone 51 (audio input device) and a foot switch 52 (operation device). The microphone 51 is an input device for performing audio recognition, which will be described later. The foot switch 52 is an operation device that is placed at an operator's feet and operated with the foot, and outputs an operation signal (for example, a signal indicating an audio input trigger or a signal to select a candidate for audio recognition) by stepping on a pedal. Note that, in this embodiment, the microphone 51 and the foot switch 52 are controlled by the input control unit 44 of the endoscopic image generation device 40, but the present invention is not limited to this embodiment, and the microphone 51 and the foot switch 52 may also be controlled via the endoscopic image processing device 60, the display device 70, and the like. In addition, in the operation part 22 of the endoscope 20, an operation device (button, switch, and the like) having the same function as the foot switch 52 may be provided.

In addition, the input device 50 can include a known input device such as a keyboard, a mouse, a touch panel, and a gaze input device as the operation device.

### Endoscopic Image Processing Device

The endoscopic image processing device 60 includes, as a hardware configuration, a processor, a main storage unit, an auxiliary storage unit, a communication unit, and the like. That is, the endoscopic image processing device 60 has a so-called computer configuration as the hardware configuration. The processor is configured by, for example, a CPU, a graphics processing unit (GPU), a field-programmable gate array (FPGA), a programmable logic device (PLD), and the like. The processor of the endoscopic image processing device 60 is an example of the processor in the endoscope system and in the medical information processing apparatus according to the embodiment of the present invention. The processor of the endoscopic image generation device 40 and the processor of the endoscopic image processing device 60 may share the functions of the processor in the endoscope system and in the medical information processing apparatus according to the embodiment of the present invention. For example, a form can be adopted in which the endoscopic image generation device 40 mainly has a function of an “endoscope processor” that generates endoscopic images, and in which the endoscopic image processing device 60 mainly has a function of a “computer-aided diagnosis (CAD) box” that performs image processing on the endoscopic images. However, in the present invention, a form different from such sharing of functions may be adopted.

For example, the main storage unit is configured by a memory such as a RAM. The auxiliary storage unit is configured by, for example, a non-transitory and tangible recording medium (memory) such as a flash memory, and stores computer-readable codes of various programs (which may include the medical information processing program according to the embodiment of the present invention or a part thereof) executed by the processor, and various kinds of data necessary for control or the like. In addition, the auxiliary storage unit may include various magneto-optical recording devices, semiconductor memories, and the like in addition to or instead of the flash memory. For example, the communication unit is configured by a communication interface connectable to a network. The endoscopic image processing device 60 is communicably connected to the endoscope information management system 100 via the communication unit.

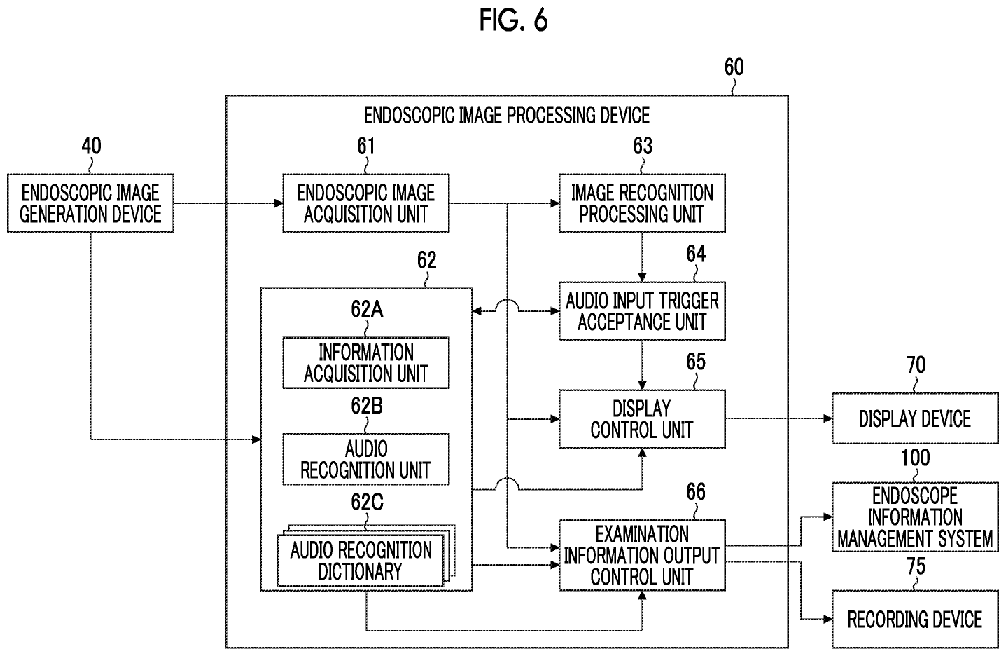

FIG. 6 is a block diagram illustrating main functions of the endoscopic image processing device 60.

As illustrated in the figure, the endoscopic image processing device 60 mainly has functions of an endoscopic image acquisition unit 61, an input information acquisition unit 62, an image recognition processing unit 63, an audio input trigger acceptance unit 64, a display control unit 65, an examination information output control unit 66, and the like. These functions are realized by the processor executing the program (which may include the medical information processing program according to the embodiment of the present invention or a part thereof) stored in the auxiliary storage unit or the like.

### Endoscopic Image Acquisition Unit

The endoscopic image acquisition unit 61 acquires an endoscopic image from the endoscopic image generation device 40. Acquisition of images can be performed in real time. That is, a plurality of medical images obtained by the image sensor 25 (image sensor) imaging the subject in chronological order can be sequentially acquired (sequentially input) in real time.

### Input Information Acquisition Unit

The input information acquisition unit 62 (processor) acquires information input via the input device 50 and the endoscope 20. The input information acquisition unit 62 includes an information acquisition unit 62A that mainly acquires input information other than the audio information, an audio recognition unit 62B that acquires the audio information and that recognizes audio input via the microphone 51, and an audio recognition dictionary 62C used for audio recognition. The audio recognition dictionary 62C may include a plurality of dictionaries with different contents (for example, dictionaries regarding site information, findings information, treatment information, and hemostasis information).

Information input to the input information acquisition unit 62 via the input device 50 includes information (for example, audio information, an audio input trigger, and information on a candidate selection operation) input via the microphone 51, the foot switch 52, or a keyboard or mouse (not illustrated). In addition, the information input via the endoscope 20 includes information on an imaging start instruction for an endoscopic image (video), an imaging instruction for a static image, and the like. As described later, in the present embodiment, a user can input the audio input trigger, perform the selection operation of the audio recognition candidate, and the like via the microphone 51 and/or the foot switch 52. The input information acquisition unit 62 acquires operation information of the foot switch 52 via the endoscopic image generation device 40.

### Image Recognition Processing Unit

The image recognition processing unit 63 (processor) performs image recognition on the endoscopic image acquired by the endoscopic image acquisition unit 61. The image recognition processing unit 63 can perform image recognition in real time.

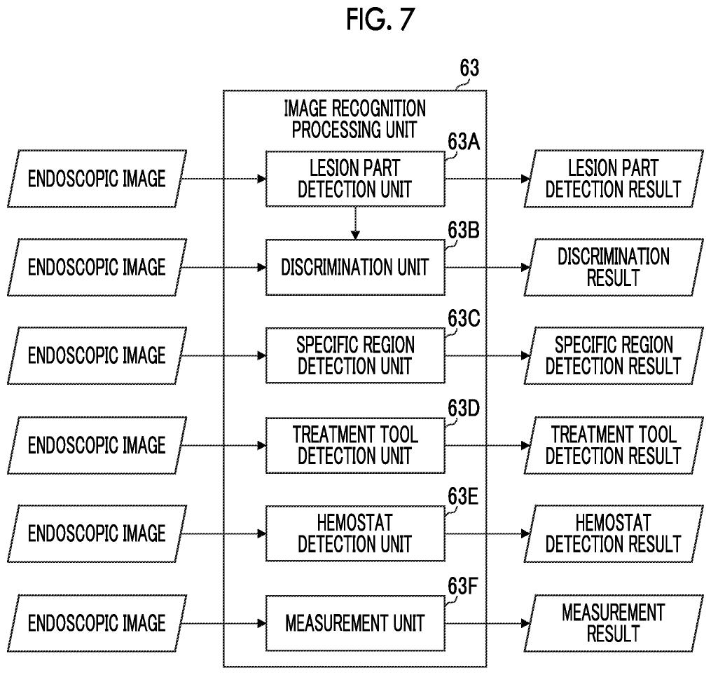

FIG. 7 is a block diagram illustrating main functions of the image recognition processing unit 63. As illustrated in the figure, the image recognition processing unit 63 has functions of a lesion part detection unit 63A, a discrimination unit 63B, a specific region detection unit 63C, a treatment tool detection unit 63D, a hemostat detection unit 63E, a measurement unit 63F, and the like. Each of these units can be used to determine “whether or not a specific subject is included in an endoscopic image”. The “specific subject” may be different depending on each unit of the image recognition processing unit 63, as described below.

The lesion part detection unit 63A detects a lesion part (lesion; an example of a “specific subject”) such as a polyp from the endoscopic image. The processing of detecting the lesion part includes processing of detecting a part with a possibility of a lesion (benign tumor, dysplasia, or the like; lesion candidate region), processing of recognizing a region after the lesion is treated (treated region) and a part with features that may be directly or indirectly associated with a lesion (erythema or the like), and the like in addition to processing of detecting a part that is definitely a lesion part.

In a case where the lesion part detection unit 63A determines that “the lesion part (specific subject) is included in the endoscopic image”, the discrimination unit 63B performs discrimination processing on the lesion part detected by the lesion part detection unit 63A. In the present embodiment, the discrimination unit 63B performs neoplastic or non-neoplastic (hyperplastic) discrimination processing on the lesion part such as a polyp detected by the lesion part detection unit 63A. Note that the discrimination unit 63B can be configured to output a discrimination result in a case where predetermined criteria are satisfied. As the “predetermined criteria”, for example, a “case where a reliability degree (depending on conditions such as exposure, degree of focus, and blurring of an endoscopic image) of the discrimination result or a statistical value thereof (maximum, minimum, average, or the like within a predetermined period) is equal to or greater than a threshold value” can be adopted, but other criteria may be used.

The specific region detection unit 63C performs processing of detecting a specific region (landmark) in the hollow organ from the endoscopic image. For example, processing of detecting an ileocecum of the large intestine or the like is performed. The large intestine is an example of a hollow organ, and the ileocecum is an example of a specific region. For example, the specific region detection unit 63C may detect a hepatic flexure (right colon), a splenic flexure (left colon), a rectosigmoid, and the like. In addition, the specific region detection unit 63C may detect a plurality of specific regions.

The treatment tool detection unit 63D performs processing of detecting a treatment tool appearing in the image from the endoscopic image, and discriminating the type of the treatment tool. The treatment tool detection unit 63D can be configured to detect a plurality of types of treatment tools such as biopsy forceps and snares. Similarly, the hemostat detection unit 63E performs processing of detecting a hemostat such as a hemostatic clip and discriminating a type of the hemostat. The treatment tool detection unit 63D and the hemostat detection unit 63E may be configured by one image recognizer.

The measurement unit 63F performs measurements (measurements of shape, dimension, and the like) of a lesion, a lesion candidate region, a specific region, a treated region, and the like.

Each unit (the lesion part detection unit 63A, the discrimination unit 63B, the specific region detection unit 63C, the treatment tool detection unit 63D, the hemostat detection unit 63E, the measurement unit 63F, and the like) of the image recognition processing unit 63 can be configured using image recognizers (trained models) configured by machine learning. Specifically, each unit described above can be configured by image recognizers (trained models) trained using a machine learning algorithm such as a neural network (NN), a convolutional neural network (CNN), AdaBoost, and random forest. In addition, as described above regarding the discrimination unit 63B, each of these units can perform an output based on the reliability degree of a final output (discrimination results, type of treatment tool, and the like) by setting a network layer configuration as necessary. In addition, each unit described above may perform image recognition for all frames of the endoscopic image, or may perform image recognition for some frames intermittently.

In the endoscope system 10, the output of the recognition result of the endoscopic image from each of these units or the output of the recognition result satisfying the predetermined criteria (threshold value or the like of the reliability degree) may be used as the audio input trigger, and a period in which such an output is performed may be used as a period in which audio recognition is executed.

In addition, instead of configuring each unit constituting the image recognition processing unit 63 using the image recognizer (trained model), some or all of the units can adopt a configuration of calculating a feature amount from the endoscopic image and performing detection or the like using the calculated feature amount.

### Audio Input Trigger Acceptance Unit

The audio input trigger acceptance unit 64 (processor) accepts an input of an audio input trigger while capturing (inputting) an endoscopic image, and sets the audio recognition dictionary 62C according to the input audio input trigger. The audio input trigger in the present embodiment is, for example, a determination result (detection result) indicating that a specific subject is included in the endoscopic image, and in this case, an output of the lesion part detection unit 63A can be used as the determination result. In addition, another example of the audio input trigger is an output of a discrimination result for the specific subject, and in this case, an output of the discrimination unit 63B can be used as the discrimination result. As still other examples of the audio input trigger, an imaging start instruction of a plurality of medical images, an input of a wake word for the microphone 51 (audio input device), an operation of the foot switch 52, an operation for another operation device (for example, colonofiberscope position determination device) connected to the endoscope system, and the like can be used. The settings of the audio recognition dictionary and the audio recognition according to these audio input triggers will be described in detail later.

### Display Control Unit

The display control unit 65 (processor) controls display of the display device 70. In the following, main display control performed by the display control unit 65 will be described.

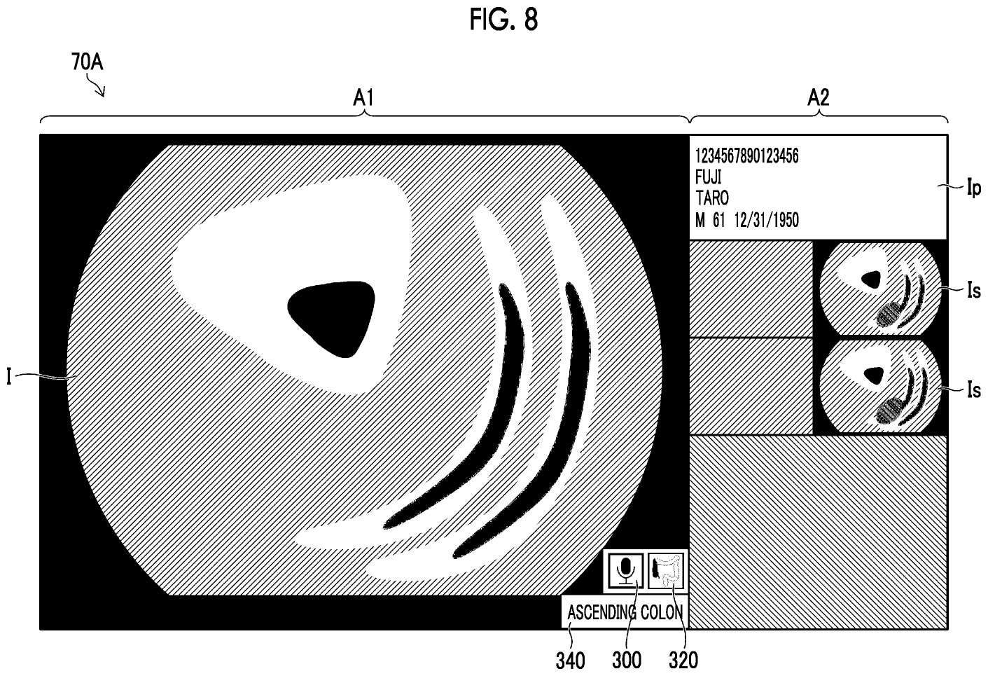

The display control unit 65 displays the image (endoscopic image) captured by the endoscope 20 on the display device 70 in real time during the examination (imaging). FIG. 8 is a diagram illustrating an example of a screen display during the examination. As illustrated in the figure, an endoscopic image I (live view) is displayed in a main display region A1 set on a screen 70A. A secondary display region A2 is further set on the screen 70A, and various kinds of information regarding the examination are displayed. In the example illustrated in FIG. 8, a case where information Ip regarding a patient and a static image Is of the endoscopic image captured during the examination are displayed in the secondary display region A2 is illustrated. For example, the static images Is are displayed in the captured order from top to bottom of the screen 70A. Note that, in a case where a specific subject such as a lesion is detected, the display control unit 65 may display the subject in an emphasized manner using a bounding box or the like.

In addition, the display control unit 65 can display, on the screen 70A, an icon 300 indicating a state of the audio recognition, an icon 320 indicating a site being imaged, and a display region 340 where a site of an imaging target (ascending colon, transverse colon, descending colon, or the like) and a result of the audio recognition are displayed in text in real time (without time delay). The display control unit 65 can acquire information on a site via image recognition from the endoscopic image, a user's input via the operation device, an external device (for example, endoscope position detecting unit) connected to the endoscope system 10, or the like, and display the information.

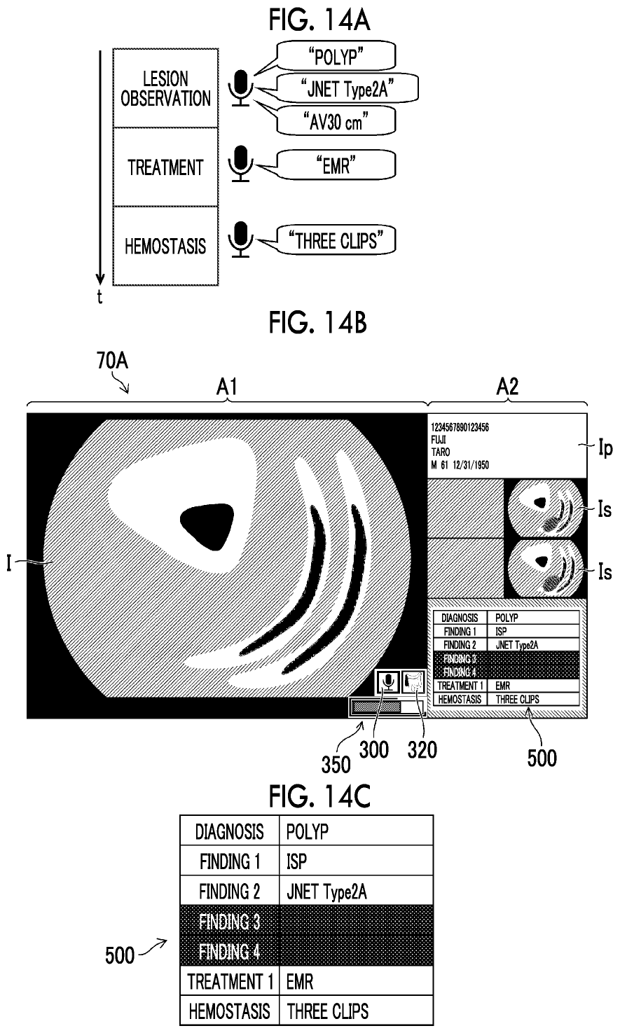

In addition, the display control unit 65 can cause the display device 70 (output device, display device) to display (output) a result of the audio recognition. The display can be performed in a lesion information input box, as will be described in detail later (refer to FIGS. 14A to 14C and the like).

### Examination Information Output Control Unit

The examination information output control unit 66 outputs examination information to the recording device 75 and/or the endoscope information management system 100. For example, the examination information includes an endoscopic image captured during an examination, a determination result for a specific subject, a result of audio recognition, information on a site input during an examination, information on a treatment name input during an examination, information on a treatment tool detected during an examination, and the like. For example, the examination information is output for each lesion or each time a specimen is collected. In this case, respective pieces of information are output in association with each other. For example, the endoscopic image in which the lesion part or the like is imaged is output in association with the information on the site being selected. In addition, in a case where a treatment is performed, the information on the selected treatment name and the information on the detected treatment tool are output in association with the endoscopic image and the information on the site. In addition, the endoscopic image captured separately from the lesion part or the like is always output to the recording device 75 and/or the endoscope information management system 100. The endoscopic image is output with the information of imaging date and time added.

### Recording Device

The recording device 75 (recording device) includes various magneto-optical recording devices or semiconductor memories, and control devices thereof, and can record endoscopic images (videos, static images), results of image recognition, results of audio recognition, examination information, report creation support information, and the like. These pieces of information may be recorded in a secondary storage unit of the endoscopic image generation device 40 or of the endoscopic image processing device 60, or in a recording device of the endoscope information management system 100.

### Audio Recognition in Endoscope System

Audio recognition in the endoscope system 10 configured as described above will be described below.

### Outline of Audio Recognition

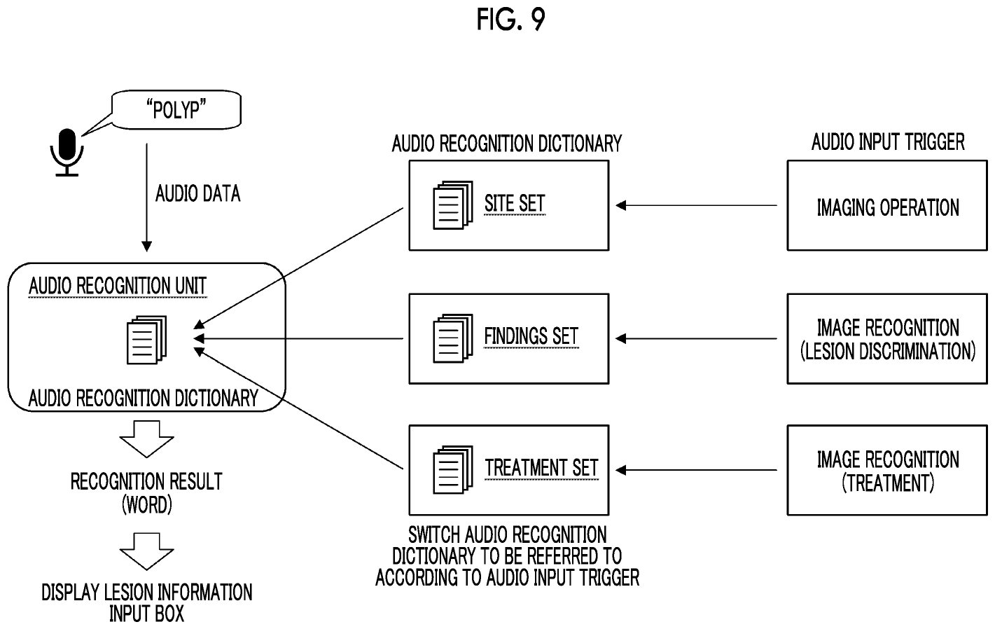

FIG. 9 is a diagram illustrating an outline of audio recognition. As illustrated in the figure, the medical information processing apparatus 80 (processor) accepts an input of an audio input trigger while capturing the endoscopic image (sequentially inputting), sets an audio recognition dictionary according to the audio input trigger in a case where the audio input trigger is input, and performs audio recognition on the audio input via the microphone 51 (audio input device) after the audio recognition dictionary is set, using the set audio recognition dictionary. As described above, the medical information processing apparatus 80 decides that an output of the detection result from the lesion part detection unit 63A, an output of the discrimination result from the discrimination unit 63B, an imaging start instruction of a plurality of medical images, a switching operation from a detection mode to a discrimination mode, an input of a wake word for the microphone 51 (audio input device), an operation of the foot switch 52, an input of an operation for an operation device connected to the endoscope system, or the like is an “input of the audio input trigger”, and performs audio recognition.

Note that the start of the audio recognition may be delayed for the settings of the audio recognition dictionary, but it is preferable that the audio recognition is started (zero delay time) immediately after the audio recognition dictionary is set.

### Settings of Audio Recognition Dictionary

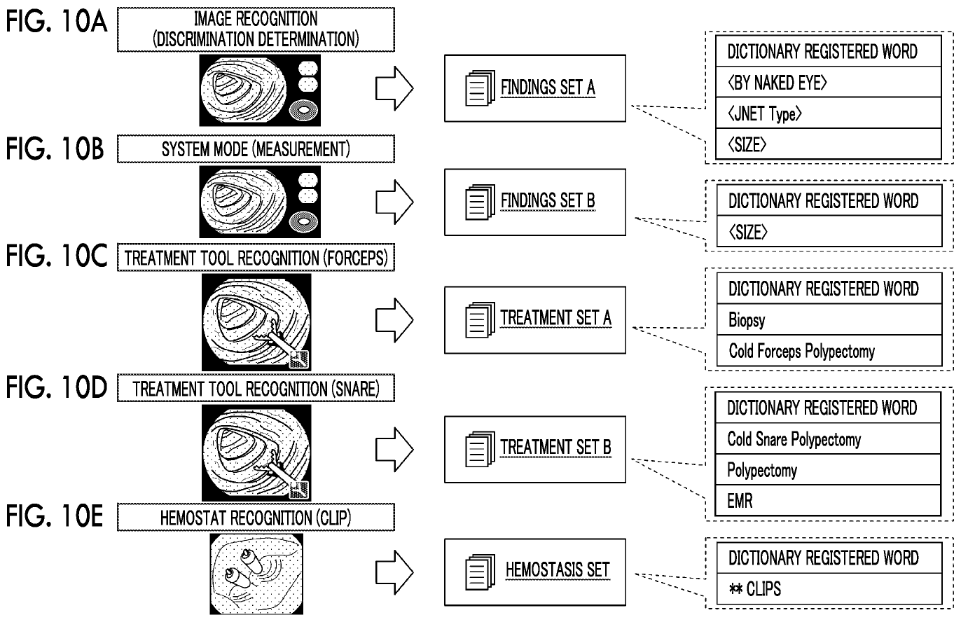

FIGS. 10A to 10E are diagrams illustrating settings of the audio recognition dictionary. In each of FIGS. 10A to 10E, a left side of an arrow indicates the audio input trigger, and a right side of an arrow indicates an example of the audio recognition dictionary set according to the audio input trigger and registered words. As illustrated in FIGS. 10A to 10E, in a case where the audio input trigger is input, the audio recognition unit 62B sets the audio recognition dictionary 62C according to the audio input trigger. For example, in a case where the discrimination unit 63B outputs a discrimination result, the audio recognition unit 62B sets a “findings set A” as the audio recognition dictionary. Note that, in addition to the examples illustrated in FIGS. 10A to 10E, the audio recognition unit 62B may set a dictionary of “sites” using an imaging operation as a trigger.

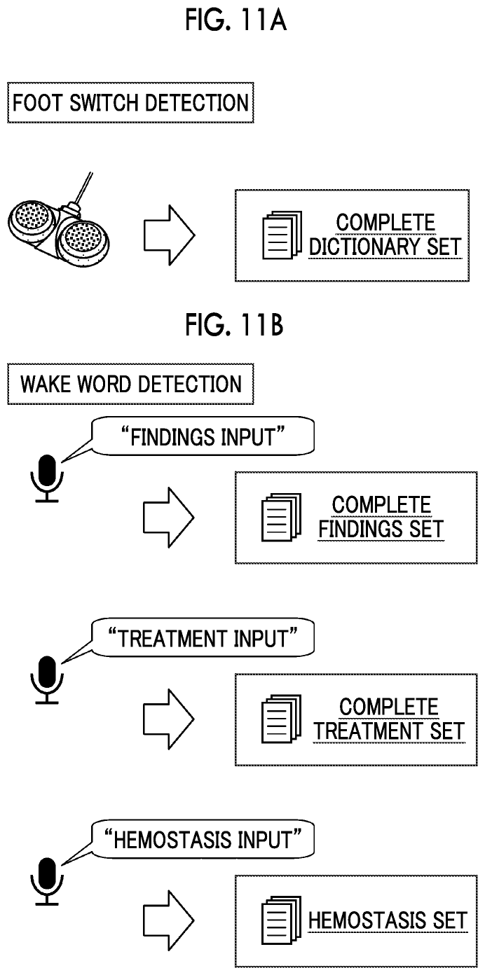

FIGS. 11A and 11B are other diagrams illustrating settings of the audio recognition dictionary. As illustrated in FIGS. 11A and 11B, the audio recognition unit 62B sets a “complete dictionary set” in a case of accepting an operation of the foot switch 52 (operation device) as the audio input trigger, and sets the audio recognition dictionary according to the contents of a wake word in a case of accepting an input of the wake word for the microphone 51 (audio input device) as the audio input trigger. Note that a “wake word” or a “wakeup word” can be defined as, for example, “a predetermined phrase for causing the audio recognition unit 62B to set the audio recognition dictionary and to start the audio recognition”.

The wake words (wakeup words) described above can be divided into two types. The two types are a “wake word regarding a report input” and a “wake word regarding imaging mode control”. The “wake word regarding a report input” is, for example, a “findings input” and a “treatment input”. After such a wake word is recognized, the audio recognition dictionary for “findings” and “treatment” is set, and in a case where a word in the dictionary is recognized, the result of the audio recognition is output. The result of the audio recognition can be associated with the image or used in a report. The association with the image and the use in the report are a form of an “output” of the result of the audio recognition, and the display device 70, the recording device 75, the storage unit of the medical information processing apparatus 80, or the recording device of the endoscope information management system 100 or the like is a form of an “output device”.

The other “wake word regarding imaging mode control” is, for example, “imaging setting” and “setting”. After such a wake word is recognized, it is possible to set a dictionary used to turn on/off or switch a light source with audio (for example, by audio recognition of words such as “white”, “LCI”, and “BLI”), or to turn on/off (for example, by audio recognition of words such as “detection on” and “detection off”) the lesion detection using an endoscope AI (recognizer using artificial intelligence). Note that the “output” and the “output device” are the same as described above for the “wake word regarding a report input”.

### Time Chart of Audio Recognition Dictionary Settings

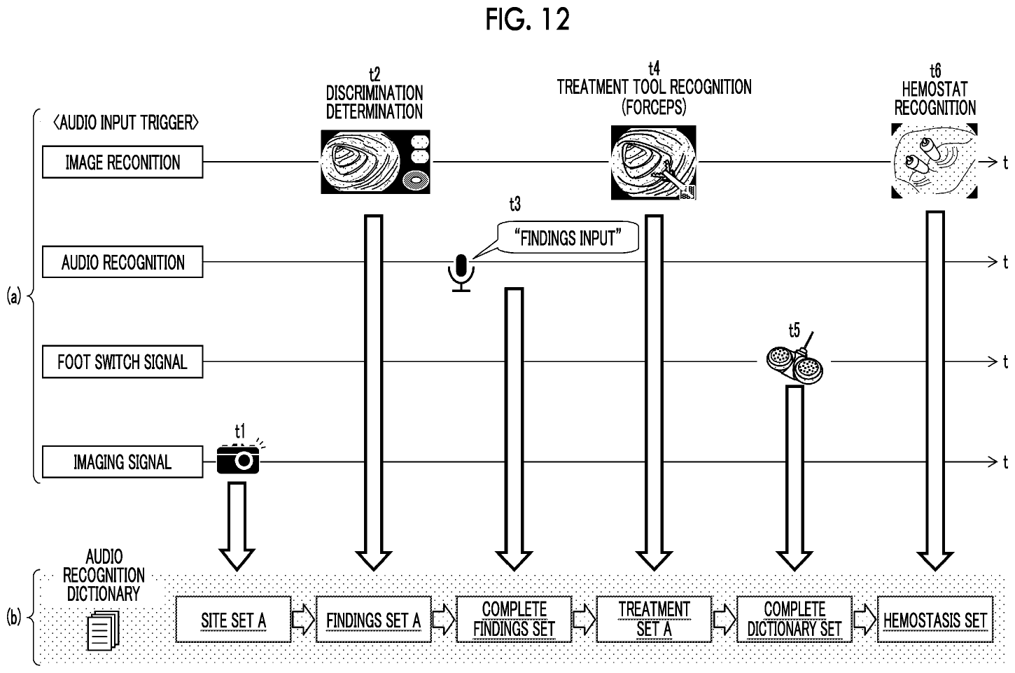

FIG. 12 is a time chart of setting the audio recognition dictionary. Note that, in FIG. 12, specific phrases that are input by audio and the recognition results thereof are not illustrated (refer to the lesion information input box of FIGS. 14A to 14C and the like). The (a) part of FIG. 12 illustrates the types of audio input triggers. In the example illustrated in the (a) part of FIG. 12, the audio input triggers are an output of a result of image recognition of the endoscopic image, an input of a wake word for the microphone 51, a signal from an operation of the foot switch 52 (operation device), and an imaging start instruction of the endoscopic image. In addition, the (b) part of FIG. 12 illustrates the audio recognition dictionaries set according to the audio input triggers. The audio recognition unit 62B sets different audio recognition dictionaries according to the flow of the examination (start of imaging, discovery of a lesion or a lesion candidate, a findings input, insertion of a treatment tool and treatment, and hemostasis). Note that the audio recognition unit 62B may set only one audio recognition dictionary 62C at a time, and may set a plurality of audio recognition dictionaries 62C at the same time. For example, the audio recognition unit 62B may set the audio recognition dictionary according to the output result of a specific one image recognizer, or may set a plurality of audio recognition dictionaries 62C according to results output from a plurality of image recognizers or to a result of a manual operation. In addition, the audio recognition unit 62B may switch the audio recognition dictionary 62C as the examination progresses.

In the endoscope system 10, image recognition (as a whole, a plurality of times of image recognition) corresponding to a plurality of types of “specific subjects” (specifically, the lesion, the treatment tool, the hemostat, and the like described above) as the determination (recognition) target can be performed by each unit of the image recognition processing unit 63, and the audio recognition unit 62B can set the audio recognition dictionary corresponding to the type of the “specific subject” determined to be “included in the endoscopic image” by any image recognition by each unit.

In addition, in the endoscope system 10, whether or not a plurality of “specific subjects” are included in the endoscopic image is determined by each unit, and the audio recognition unit 62B can set the audio recognition dictionary corresponding to the specific subject determined to be “included in the endoscopic image” among the plurality of “specific subjects”. As a case where a plurality of “specific subjects” are included in the endoscopic image, for example, a case where a plurality of lesion parts are included, a case where a plurality of treatment tools are included, a case where a plurality of hemostats are included, and the like are considered.

Note that, for some image recognition among a plurality of times of image recognition by the respective units, the audio recognition dictionary may be set according to the type of the “specific subject”.

### Audio Recognition

The audio recognition unit 62B performs audio recognition on the audio input to the microphone 51 (audio input device) after the audio recognition dictionary is set, using the set audio recognition dictionary (illustration is omitted in FIG. 12). It is preferable that the display control unit 65 displays the result of the audio recognition on the display device 70.

In the present embodiment, the audio recognition unit 62B can perform audio recognition for site information, findings information, treatment information, and hemostasis information. Note that, in a case where there are a plurality of lesions or the like, a series of processing (acceptance of audio input triggers, setting audio recognition dictionaries, and audio recognition in a cycle from imaging start to hemostasis) can be repeated for each lesion or the like. As described below, the audio recognition unit 62B and the display control unit 65 display an audio information input box during audio recognition.

### Audio Recognition and Words Displayed as Results

In the endoscope system 10, in the audio recognition, the audio recognition unit 62B and the display control unit 65 (processor) can recognize only the registered words that are registered in the set audio recognition dictionary, and display (output) the result of the audio recognition for the registered word on the display device 70 (output device, display device) (adaptive audio recognition). According to this form, only the registered words registered in the set audio recognition dictionary are audio-recognized, so that the recognition accuracy can be improved. Note that, in such adaptive audio recognition, the registered words of the audio recognition dictionary may be set so that the wake word is not recognized, or the registered words may be set to include the wake word.

In addition, in the endoscope system 10, in the audio recognition, the audio recognition unit 62B and the display control unit 65 (processor) can recognize the registered words that are registered in the set audio recognition dictionary and specific words, and display (output) the result of the audio recognition for the registered word among the recognized words on the display device 70 (output device, display device) (non-adaptive audio recognition). Note that an example of the “specific word” is a wake word for the audio input device, but the “specific word” is not limited thereto.

Note that, in the endoscope system 10, which of the above forms (adaptive audio recognition, non-adaptive audio recognition) is used to perform audio recognition and to display the result can be set on the basis of an instruction input from the user via the input device 50, the operation part 22, or the like.

### Notification of Audio Recognition State to User

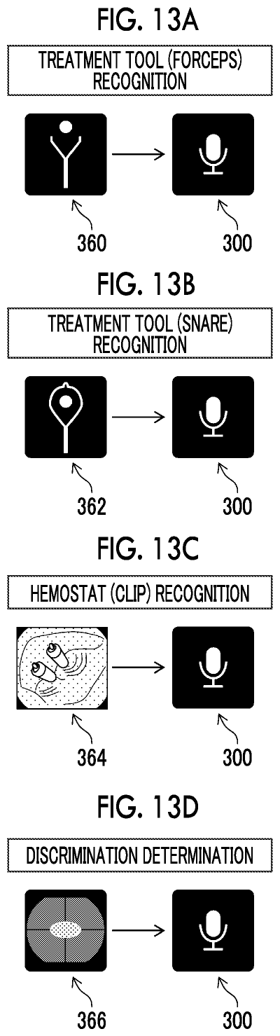

Note that, in the endoscope system 10, it is preferable that the display control unit 65 (processor) notifies the user that the audio recognition dictionary is set (the fact that the audio recognition dictionary is set and which dictionary is set) and that the audio recognition is possible. As illustrated in FIGS. 13A to 13D, the display control unit 65 can perform notification by switching icons displayed on the screen. In the examples illustrated in FIGS. 13A to 13D, the display control unit 65 displays the icon indicating the image recognizer that is operating (or displaying the recognition result on the screen) among the respective units of the image recognition processing unit 63 on the screen such as the screen 70A, and switches the display to a microphone icon 300 in a case where the image recognizer recognizes the specific subject (audio input trigger) and an audio recognition period is reached, thereby notifying the user (refer to FIGS. 8, 13A to 13D, 14B and 31A).

Specifically, FIGS. 13A and 13B illustrate a state where the treatment tool detection unit 63D is operating, but the specific subjects as the recognition target are different (forceps, snare). Therefore, the display control unit 65 displays different icons 360 and 362, and switches to the microphone icon 300 in a case where the forceps or the snare is actually recognized, thereby notifying the user that the audio recognition is possible. Similarly, FIGS. 13C and 13D illustrate states in which the hemostat detection unit 63E and the discrimination unit 63B are operating, respectively. The display control unit 65 displays an icon 364 or an icon 366, and switches to the microphone icon 300 in a case where the hemostat or the lesion is recognized, thereby notifying the user that the audio recognition is possible. The display control unit 65 may display a plurality of icons in a case where a plurality of audio recognition dictionaries 62C are set.

Note that the icon described above is one form of “type information” indicating the type of the audio recognition dictionary.

Through such notification, the user can easily ascertain that a specific image recognizer is operating and that a period in which audio recognition is possible is reached. Note that the display control unit 65 may display and switch the icon according to not only the operation situation of each unit of the image recognition processing unit 63 but also an operation situation and an input situation of the microphone 51 and/or the foot switch 52.

Note that it is possible to notify of the audio recognition state using distinguishable display of the lesion information input box in addition to or instead of directly notifying of the audio recognition state using icons (refer to FIGS. 14A to 14C and the like).

### Display of Lesion Information Input Box

FIGS. 14A to 14C are diagrams illustrating the audio input, the audio recognition, and the display of the lesion information input box. FIG. 14A illustrates an example of a flow of an audio input associated with the examination. In the example of FIG. 14A, for one lesion, the lesion observation (diagnosis, findings input), the treatment, and the hemostasis are performed, and along with this, the audio input and the audio recognition are executed. Such processing can be repeated for each lesion. FIG. 14B is a diagram illustrating a state in which a lesion information input box 500 is displayed on a screen of the display device 70 according to the audio input and the audio recognition. As illustrated in FIG. 14B, the audio recognition unit 62B and the display control unit 65 can display the lesion information input box 500 on the same display screen as the endoscopic image. It is preferable that the audio recognition unit 62B and the display control unit 65 display the lesion information input box 500 in a region different from the image display region so as not to obstruct the observation of the endoscopic image.

FIG. 14C is an enlarged diagram of the lesion information input box 500. The lesion information input box 500 is a region where item information indicating an item recognized using the audio recognition dictionary, and a result of the audio recognition corresponding to the item information are displayed in association with each other. In the present embodiment, the “item information” is diagnosis, findings (findings 1 to 4), treatment, and hemostasis. It is preferable that the item information includes at least one of the items, and a configuration may be adopted in which a plurality of inputs can be made for a specific item. In addition, it is preferable that the audio recognition unit 62B and the display control unit 65 display the item information and the result of the audio recognition in chronological order of the processing (diagnosis, findings, treatment, hemostasis) as illustrated in the examples of FIGS. 14A to 14C.

In the example illustrated in FIG. 14C, the “results of the audio recognition” are “polyp” for “diagnosis”, “ISP (note: a form of polyp)” for “finding 1”, “endoscopic mucosal resection (EMR)” for “treatment”, and “three clips” (clip: a form of hemostat) for “hemostasis”.

In the examples illustrated in FIGS. 14A to 14C, the audio recognition unit 62B and the display control unit 65 display, in a distinguishable manner, items “finding 3” and “finding 4” with no input in the lesion information input box 500 by changing the color (an example of distinctive power) from the already inputted region. Thereby, the user can easily ascertain the item information that has been input and the item information that has not been input.

Note that, as will be described in detail later, it is preferable that the audio recognition unit 62B and the display control unit 65 display the lesion information input box 500 during a period in which the audio input is accepted (not displaying constantly, but displaying for a limited time). Thereby, the result of the audio recognition can be presented to the user in an easy-to-understand format without hindering the visibility of other information displayed on the screen of the display device 70.

### Basic Display Operation of Lesion Information Input Box

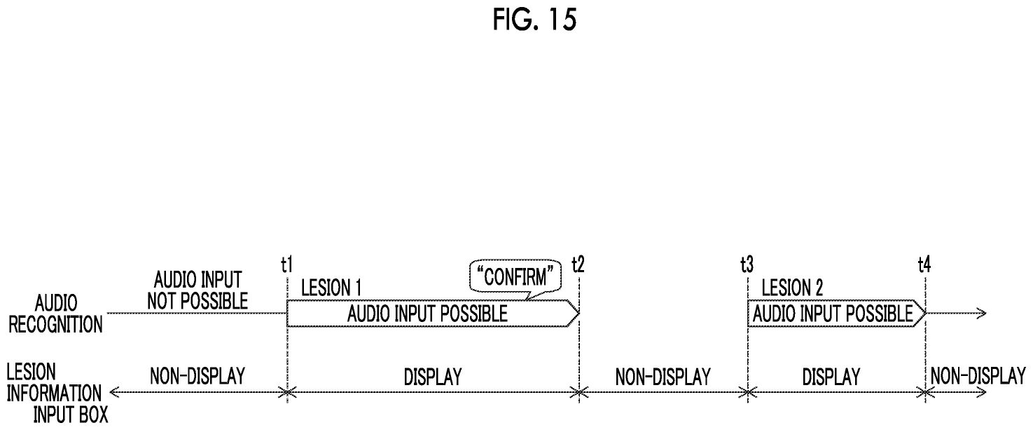

FIG. 15 is a diagram illustrating a basic display operation of the lesion information input box. As illustrated in FIG. 15, the display control unit 65 displays the lesion information input box during a period in which the audio recognition dictionary is set so that the audio input is possible (display period after the audio recognition dictionary is set). The display control unit 65 may set a period with a length depending on the type of the audio input trigger, as the display period. Note that it is preferable that the input to and the display of the lesion information input box are performed for each lesion (an example of the subject) (display is performed for each of lesions 1 and 2 in FIG. 15).

The display control unit 65 ends the display of the lesion information input box in a case where the display period has elapsed (it is preferable to display the lesion information input box temporarily rather than displaying the lesion information input box constantly), but may end the display of the lesion information input box without waiting for the elapse of the display period. For example, the display control unit 65 may accept confirmation information indicating confirmation of the audio recognition for each lesion, end, in a case where the confirmation information is accepted, the display of the item information and the result of the audio recognition for the subject, and accept the input of the audio input trigger for another subject. The user can input the confirmation information using the operation via the foot switch 52, the operation via the other input device 50, or the like.

### Display of Lesion Information Input Box: Aspect 1

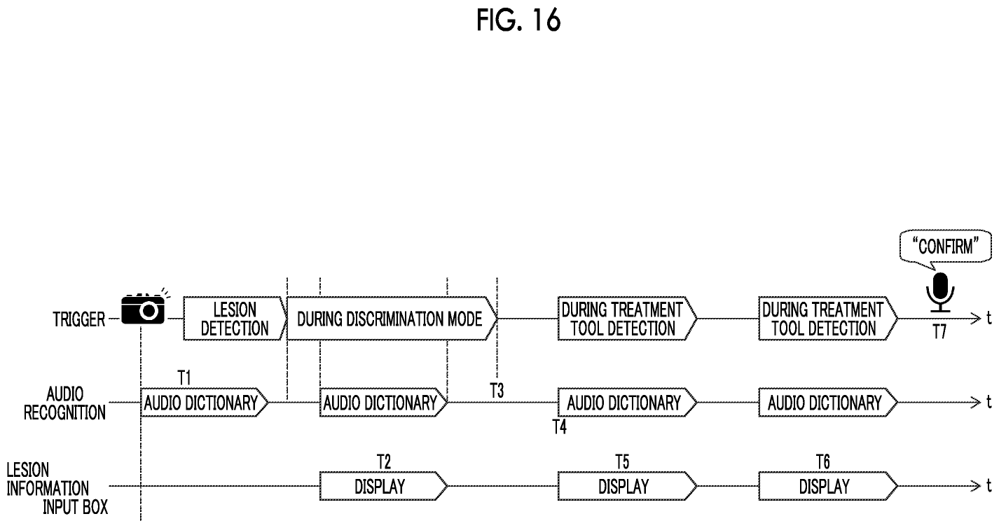

Specific display modes of the lesion information input box will be described below. FIG. 16 is a diagram illustrating a display sequence (Aspect 1) of the lesion information input box.

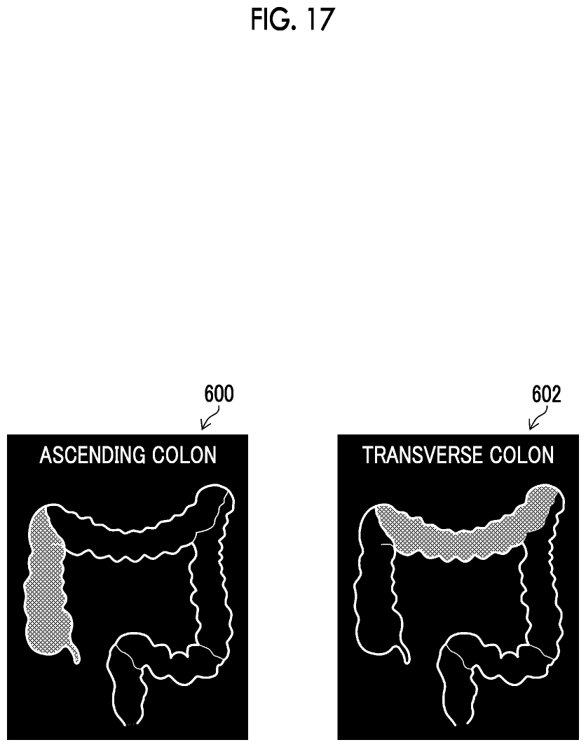

The audio recognition unit 62B sets the audio recognition dictionary (here, dictionary for site selection) using the imaging start instruction of the endoscopic image as the audio input trigger, during a period T1. For example, the display control unit 65 displays an icon 600 indicating the ascending colon and an icon 602 indicating the transverse colon on the screen 70A of the display device 70 as in FIG. 17 (diagram illustrating a display example of options of the site). The user can select a site by an audio input via the microphone 51 or by operating the foot switch 52, and the display control unit 65 continues to display the selection result until the site is changed (refer to the icon 320 in FIG. 8).

Regarding the display of the site described above, the audio recognition unit 62B and the display control unit 65 may constantly display icons (the icons 600 and 602 in FIG. 17 or the icon 320 or the like in FIG. 8; site schema diagram) indicating the sites on the screen 70A, and accept the selection of the site by the user only during the period in which the audio recognition dictionary is set on the basis of the imaging start instruction. In this case, the display control unit 65 may display, in an emphasized manner (enlargement, coloration, or the like), the icon as the result of selecting the site.

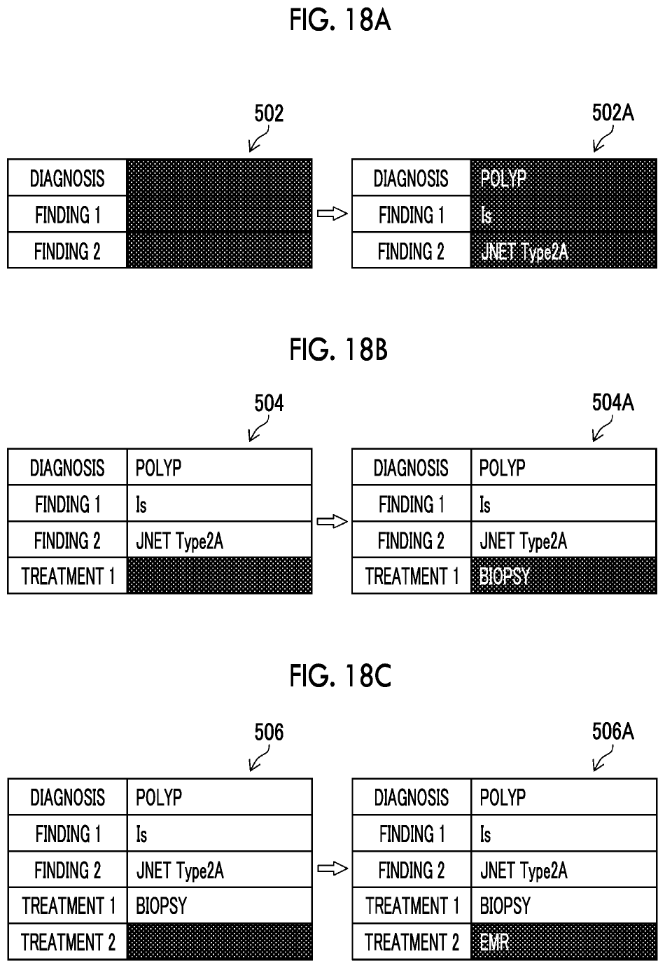

During a period T2, the audio recognition unit 62B sets the audio recognition dictionary using the output of the discrimination result of the discrimination unit 63B as the audio input trigger. The audio recognition unit 62B and the display control unit 65 display “diagnosis” and “findings 1 and 2” as illustrated in a lesion information input box 502 in FIG. 18A (diagram illustrating transition of the display of the lesion information input box) on the screen 70A or the like (refer to examples of FIGS. 14A to 14C), and display the result as illustrated in a lesion information input box 502A in a case where the audio recognition is performed for the display items. As illustrated in FIG. 18A, the item with no input can be displayed in a distinguishable manner by changing the color (the same applies to the examples described below).

Returning to FIG. 16, a period T3 is a wake word detection period, and the audio recognition dictionary for report creation support (for lesion information input box) is not set. A period T4 is a period in which the audio recognition dictionary for the report creation support (here, the audio recognition dictionary for treatment tool detection) is set.

A period T5 is a period in which the lesion information input box is displayed corresponding to the period T4. The audio recognition unit 62B and the display control unit 65 display a lesion information input box 504 in which the item of “treatment 1” has not been input as illustrated in FIG. 18B, and display “biopsy” for “treatment 1” as in a lesion information input box 504A in a case where the audio input is performed.

Returning to FIG. 16 again, a period T6 is a period in which the audio recognition dictionary for treatment tool detection is set, similar to the period T5. The audio recognition unit 62B and the display control unit 65 display a lesion information input box 506 in which the item of “treatment 2” has not been input as illustrated in FIG. 18C, and display “EMR” for “treatment 2” as in a lesion information input box 506A in a case where the audio input is performed. Note that, normally, a plurality of treatment names are not input for the same lesion. Therefore, the audio recognition unit 62B and the display control unit 65 can overwrite and update the contents of “treatment” in cases other than “biopsy”.

### Display of Lesion Information Input Box: Modification Example of Aspect 1

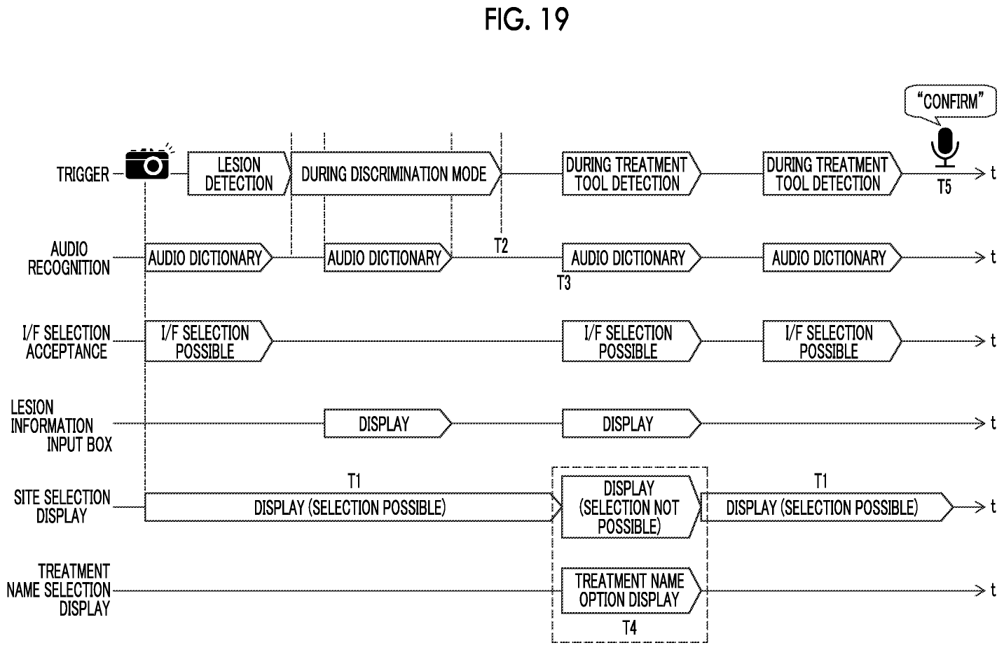

Another display mode (modification example of Aspect 1) of the lesion information input box will be described. FIG. 19 is a diagram illustrating a display sequence in a modification example. In this modification example, similarly to Aspect 1, the discrimination result output of the discrimination unit 63B serves as the audio input trigger. Note that the selection of the site and the display of the selection result (refer to FIG. 17) are performed in the same manner as in Aspect 1. In addition, during a period of “interface (I/F) selection allowed”, the input control unit 44 (processor) accepts an input from an operation device other than the microphone 51 (audio input device), such as the foot switch 52.

In the example of FIG. 19, a period T1 is a period in which candidates for the site are displayed and selection is accepted, as illustrated in FIG. 17. A period T2 is a wake word detection period, and the audio recognition dictionary for report creation support (for lesion information input box) is not set. A period T3 is a period in which the audio recognition dictionary for the report creation support (here, the audio recognition dictionary for treatment tool detection) is set. A period T4 is a period in which selection of the treatment name is accepted as described below.

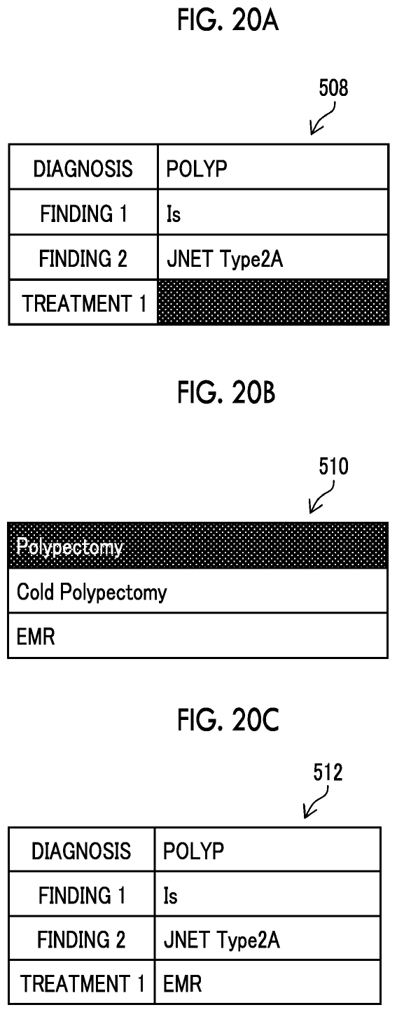

FIGS. 20A to 20C are diagrams illustrating states of the display of the lesion information input box during a period T4. The audio recognition unit 62B and the display control unit 65 display a lesion information input box 508 in which the item of “treatment 1” has not been input, and candidates 510 for “treatment 1” on the screen 70A or the like as illustrated in FIGS. 20A and 20B. The user can perform a selection operation of the treatment name using an operation device such as the microphone 51 or the foot switch 52, and in a case where the selection is made, the audio recognition unit 62B and the display control unit 65 display “EMR” for “treatment 1” as in a lesion information input box 512 illustrated in FIG. 20C.

### Display of Lesion Information Input Box: Aspect 2

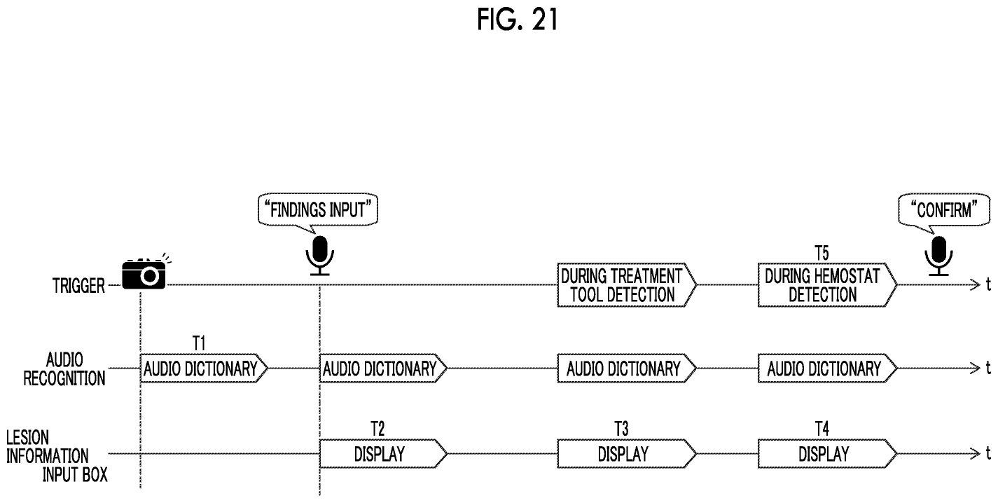

Still another display mode (Aspect 2) of the lesion information input box will be described. FIG. 21 is a diagram illustrating a display sequence in Aspect 2. In Aspect 2, an audio input (phrase of “findings input”) via the microphone 51 serves as the audio input trigger. In a period T1, similarly to the period T1 in FIG. 16, the audio recognition dictionary for site selection is set using the imaging start instruction as the audio input trigger, and the selection result is displayed.

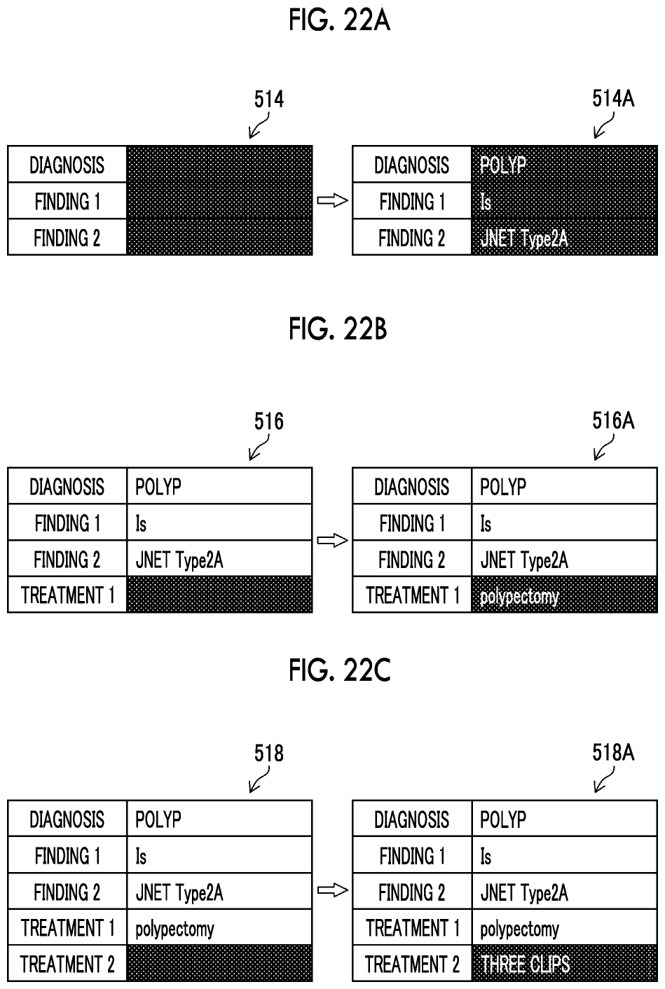

During a period T2, the audio recognition dictionary (for example, “findings set A” illustrated in FIG. 10A) is set using an input of the phrase of “findings input” as the audio input trigger. The audio recognition unit 62B and the display control unit 65 display a lesion information input box 514 in which the items of “diagnosis”, “finding 1”, and “finding 2” have not been input as illustrated in FIG. 22A, and respectively display “polyp”, “Is”, “JNET Type2A” for “diagnosis”, “finding 1”, and “finding 2” as in a lesion information input box 514A in a case where the audio input is performed.

During a period T3, the audio recognition dictionary is set using the detection of the treatment tool as the audio input trigger. The audio recognition unit 62B and the display control unit 65 display a lesion information input box 516 in which the item of “treatment 1” has not been input as illustrated in FIG. 22B, and display “polypectomy” for “treatment 1” as in a lesion information input box 516A in a case where the audio input is performed.

Similarly, during periods T4 and T5, the audio recognition dictionary is set using the detection of the hemostasis as the audio input trigger. The audio recognition unit 62B and the display control unit 65 display a lesion information input box 518 in which the item of “hemostasis 1” has not been input as illustrated in FIG. 22C, and display “three clips” for “hemostasis 1” as in a lesion information input box 518A in a case where the audio input is performed. In this manner, in Aspect 2, each time the audio input and the audio recognition are performed, items and audio recognition results to be displayed in the lesion information input box are increased.

Note that it is preferable that, in a case of performing discrimination recognition and performing hemostasis recognition, the audio recognition unit 62B sets the audio recognition dictionary during a period in which the reliability degree of the output of the recognition result or the statistical value thereof is equal to or greater than the threshold value (an example of a reference value). A situation in which the reliability degree or the like temporarily exceeds (or falls below) the threshold value can be avoided by providing a temporal width to the timing of determining the threshold value.

### Display of Lesion Information Input Box: Aspect 3

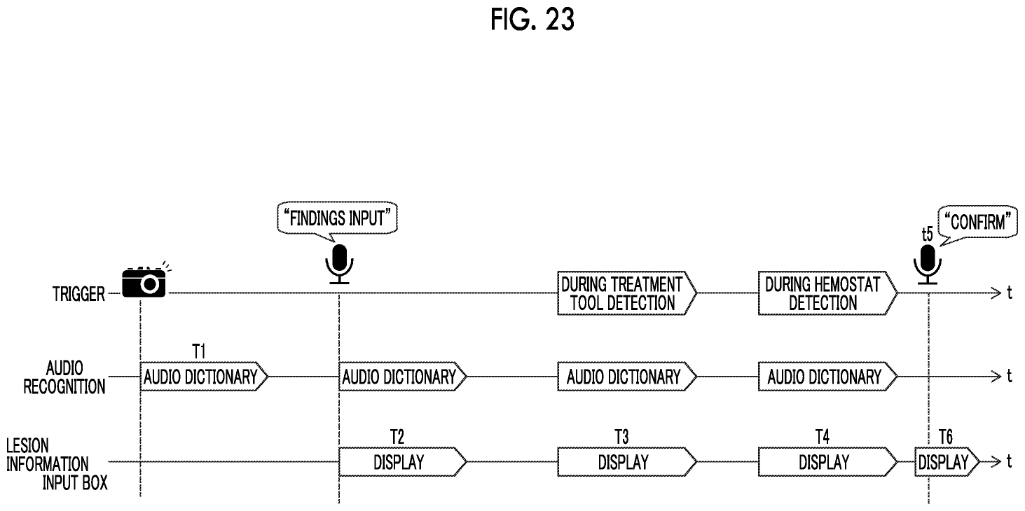

Still another display mode (Aspect 3) of the lesion information input box will be described. FIG. 23 is a diagram illustrating a display sequence in Aspect 3. Also in Aspect 3, an audio input (phrase of “findings input”) via the microphone 51 serves as the audio input trigger during a period T2. During a period T1, similarly to the period T1 in FIGS. 16 and 21, the audio recognition dictionary for site selection is set using the imaging start instruction as the audio input trigger, and the selection result is displayed.

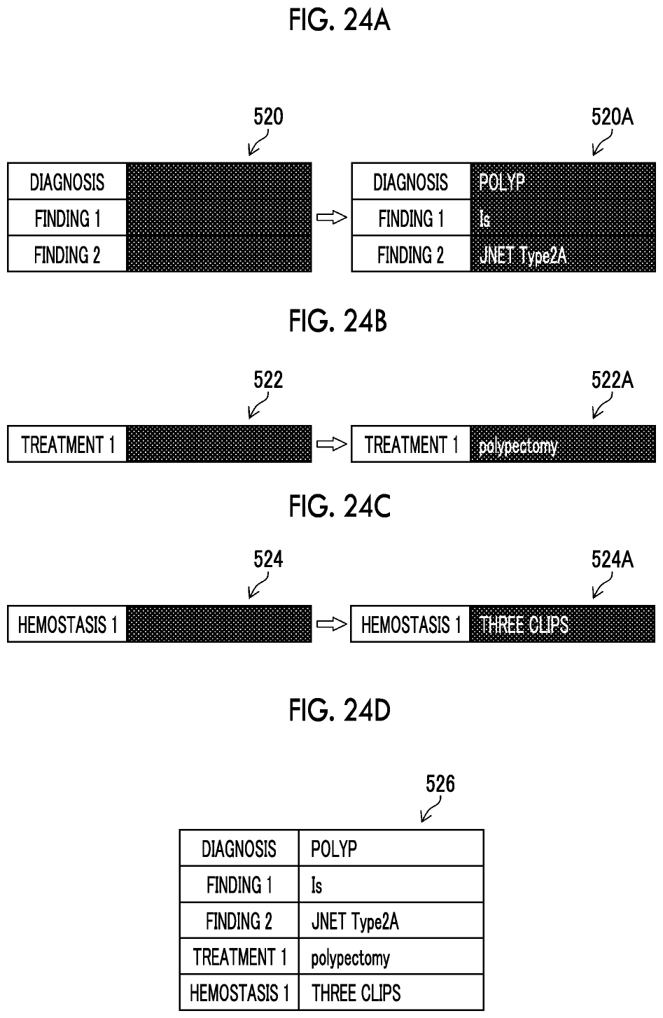

During a period T2, the audio recognition unit 62B and the display control unit 65 display a lesion information input box 520 in which the items of “diagnosis”, “finding 1”, and “finding 2” have not been input as illustrated in FIG. 24A, and respectively display “polyp”, “Is”, “JNET Type2A” for “diagnosis”, “finding 1”, and “finding 2” as in a lesion information input box 520A in a case where the audio input is performed.

During a period T3, the audio recognition unit 62B and the display control unit 65 display a lesion information input box 522 in which the item of “treatment 1” has not been input as illustrated in FIG. 24B, and display “polypectomy” for “treatment 1” as in a lesion information input box 522A in a case where the audio input is performed.

Similarly, during a period T4, the audio recognition unit 62B and the display control unit 65 display a lesion information input box 524 in which the item of “hemostasis 1” has not been input as illustrated in FIG. 24C, and display “three clips” for “hemostasis 1” as in a lesion information input box 524A in a case where the audio input is performed.

In a case where the audio of the phrase of “confirm” is input via the microphone 51 at time point t5, the audio recognition unit 62B and the display control unit 65 display a lesion information input box 526 including the audio recognition results of the display items that have been accepted as illustrated in FIG. 24D, only during a period T6. In this manner, in Aspect 3, only the display item as the target of the audio recognition is displayed, and in a case where a confirmation operation is performed, the results are displayed all at once. Thereby, it is possible to reduce a display space of the lesion information input box.

### Other Display Modes of Lesion Information Input Box

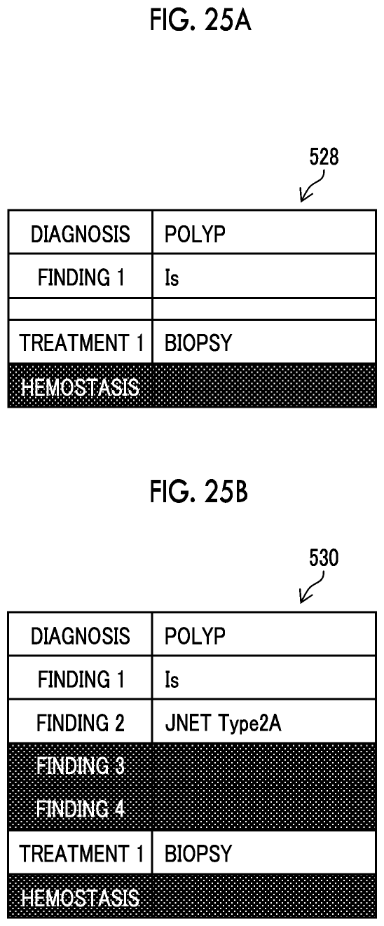

FIGS. 25A and 25B are diagrams illustrating another display mode (variation) of the lesion information input box. FIG. 25A is an example of hiding the display items (“finding 2”, “finding 3”, “finding 4”) with no input (however, “hemostasis” as the item information that can be input is displayed), and FIG. 25B is an example of displaying all items of the item information regardless of whether the items have not been input or have been input (items that have not been input and items that have already been input are displayed in a distinguishable manner by changing colors; the same applies to other figures).

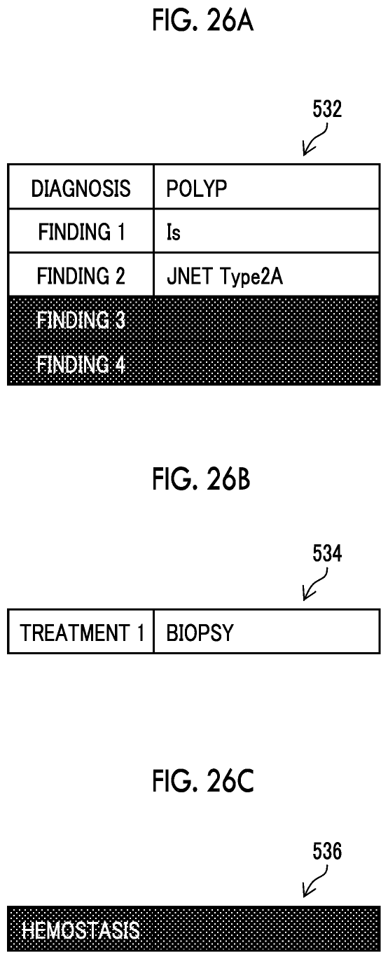

FIGS. 26A to 26C are diagrams illustrating another display mode of the lesion information input box. The mode illustrated in FIGS. 26A to 26C is a mode in which only the display items that can be input and the audio recognition results for these items are displayed according to the results of the image recognition (or the operating image recognizer). Specifically, as illustrated in FIG. 26A, the audio recognition unit 62B and the display control unit 65 display only the display items “diagnosis” and “findings 1 to 4” in a lesion information input box 532 during “discrimination” (period in which the discrimination unit 63B outputs the result). Note that, in the example of FIG. 26A, since the items of findings 3 and 4 have not been input, the items are displayed in a distinguishable manner by changing the color from that of the items that have already been input.

On the other hand, the audio recognition unit 62B and the display control unit 65 display only the display item “treatment 1” and the result for the item in a lesion information input box 534 as illustrated in FIG. 26B during “treatment”, and display only the display item “hemostasis” in a lesion information input box 536 as illustrated in FIG. 26C during “hemostasis” (the item of hemostasis has not been input, and is displayed in a distinguishable manner by changing the color from that of the items that have already been input). According to such a mode, it is possible to reduce a display space of the lesion information input box.

### Display Mode (Variation) of Lesion Information Input Box

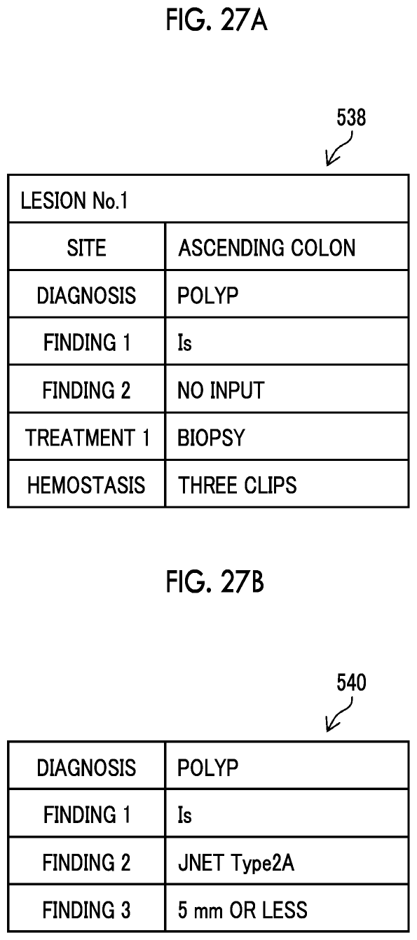

FIGS. 27A and 27B are diagrams illustrating still another display mode of the lesion information input box. In the present embodiment, as in a lesion information input box 538 illustrated in FIG. 27A, a serial number of the lesion may be set, input, and displayed in the lesion information input box. In addition, for the display item of “site”, the selected site may be input and displayed. In addition, for the item that has not been input (“finding 2” in FIG. 27A), information indicating that no input is made, such as “no input” or “blank”, may be displayed. In addition, as in a lesion information input box 540 illustrated in FIG. 27B, a display item of “finding 3” may be provided in the lesion information input box. In the display items of “findings” (findings 1 to 3”, information such as “diagnosis”, “by naked eye”, “JNET”, and “size” can be input.

### Lesion Information Input Box for Plurality of Treatments

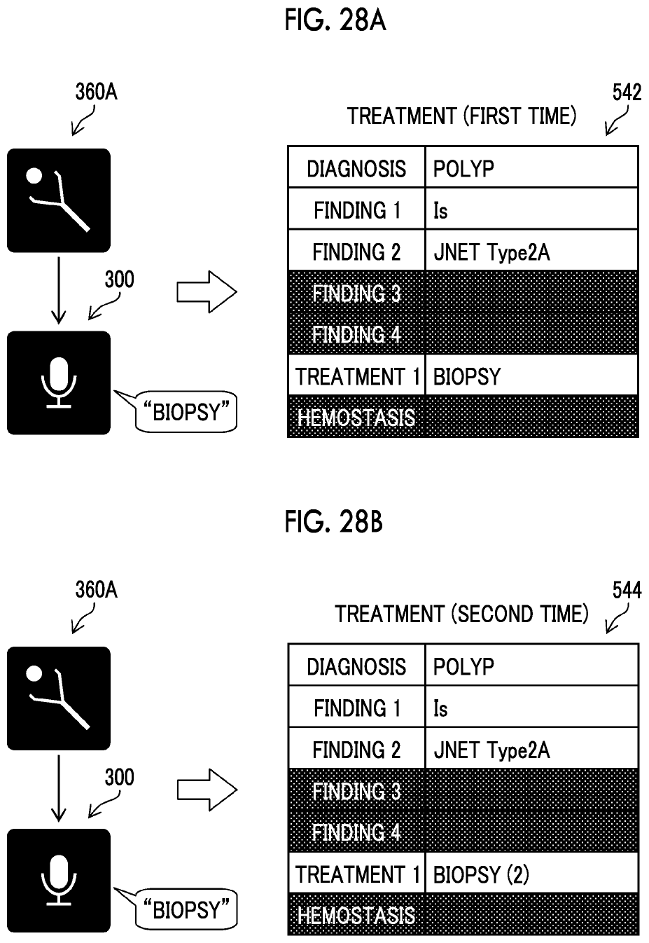

In the examination using the endoscope, a plurality of treatments may be performed on one lesion. In this case, a plurality of inputs may be performed in the lesion information input box, or may be overwritten. FIG. 28A is a diagram illustrating a state of the input to a lesion information input box 542 in a case where a first treatment is performed. In this case, the audio recognition unit 62B and the display control unit 65 switch a forceps icon 360A to the microphone icon 360 and display the icon 360A in a case where the forceps are recognized and the audio recognition is possible. In this state, in a case where the user utters “biopsy”, the audio recognition unit 62B and the display control unit 65 display “biopsy” in “treatment 1”.

FIG. 28B is a diagram illustrating a state of the input in a case where a second treatment is performed. In a case where the user utters “biopsy”, the audio recognition unit 62B and the display control unit 65 display “biopsy (2)” in “treatment 1” to indicate that the treatment is the second biopsy.

### Options of Findings Input

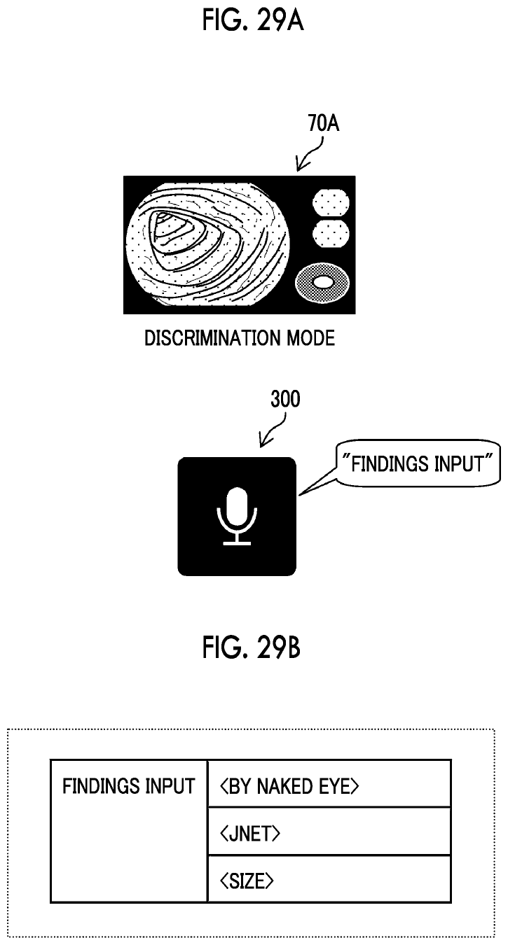

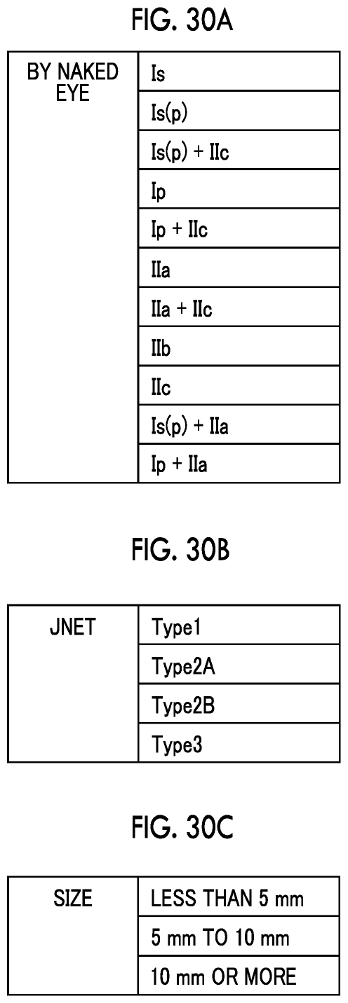

FIGS. 29A to 30C are diagrams illustrating options of the findings input (registered contents of the audio recognition dictionary for “findings”). As illustrated in FIG. 29A, in a case where the discrimination result is output, the microphone icon 300 is displayed on the screen 70A, the audio recognition dictionary for the findings input is set, and a state in which the audio recognition regarding the findings is possible is assumed. In this case, as illustrated in FIG. 29B, the items to be input in “findings” can be classified into “by naked eye”, “JNET”, and “size”. In each item of the audio recognition dictionary, the contents illustrated in FIGS. 30A to 30C are registered, and the audio recognition is possible.

### Screen Display of Remaining Time

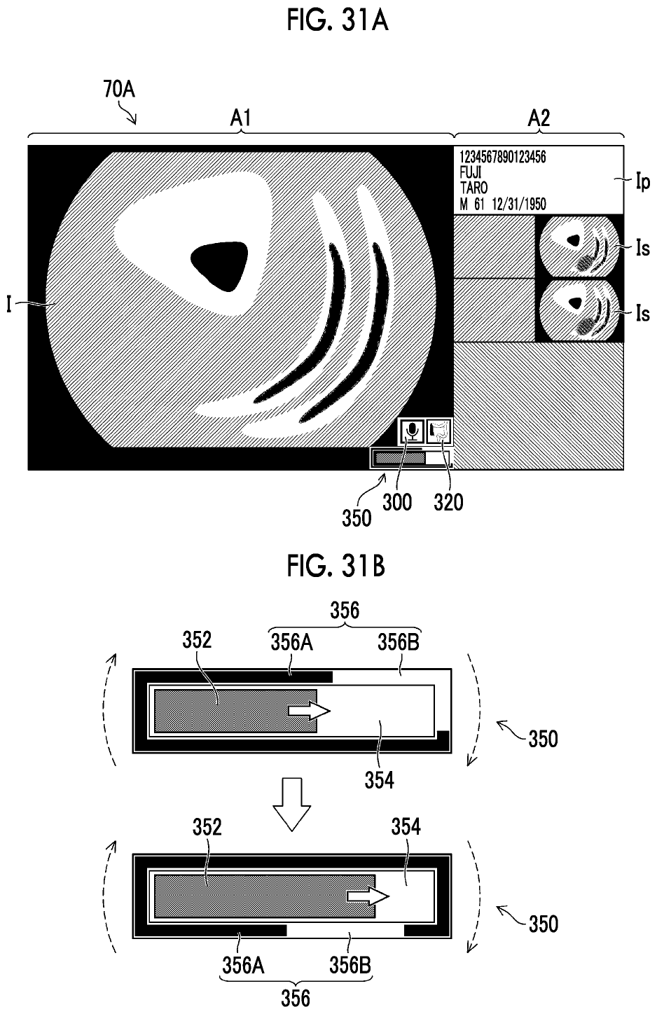

The audio recognition unit 62B and the display control unit 65 may display the remaining time (remaining time of the audio recognition period) of the display period of the lesion information input box on the screen of the display device 70. FIGS. 31A and 31B are diagrams illustrating examples of the screen display of the remaining time. FIG. 31A is an example of the display on the screen 70A, and a remaining time meter 350 is displayed. In addition, FIG. 31B is an enlarged diagram of the remaining time meter 350. In the remaining time meter 350, a hatched region 352 is lengthened as the time elapses, and a plain region 354 is shortened as the time elapses. In addition, a frame 356 consisting of a black background region 356A and a white background region 356B rotates around these regions to call the user's attention. The audio recognition unit 62B and the display control unit 65 may rotate and display the frame 356 in a case where it is detected that the audio is input.

Note that the audio recognition unit 62B and the display control unit 65 may output the remaining time using numbers or audio. Note that it may be specified that “the remaining time is zero in a case where the screen display of the microphone icon 300 (refer to FIGS. 8, 13A to 13D, 14B and 31A) disappears”.

### End of Display of Lesion Information Input Box (Summary)

There are several possible conditions for ending the display of the lesion information input box. The audio recognition unit 62B and the display control unit 65 may end the display in a case where the display period of the lesion information input box has elapsed, or may end the display of the lesion information input box in a case where the audio recognition dictionary display period ends. The display period may have a length depending on the type of the audio input trigger. In addition, regardless of the elapse of the display period, the display may be ended in a case where a state in which a specific subject is recognized ends (associated with the output of the recognizer), or the display may be ended in a case where a confirmation operation is performed.

### Record of Report Creation Support Information

In a case where the audio recognition is performed, the examination information output control unit 66 (processor) can associate the endoscopic image (a plurality of medical images) with the contents (item information and results of the audio recognition) of the lesion information input box, and record the endoscopic image and the contents in the recording device such as the recording device 75, the storage unit of the medical information processing apparatus 80, and the endoscope information management system 100. The examination information output control unit 66 may further associate the endoscopic image in which a specific subject is shown and the determination result (that the specific subject is shown in the image) of the image recognition, and record the endoscopic image and the determination result. The examination information output control unit 66 may perform recording according to the user's operation on the operation device (microphone 51, foot switch 52, or the like), or may perform recording automatically without depending on the user's operation (perform recording at predetermined intervals, recording by the operation of “confirm”, or the like). With such recording, in the endoscope system 10, the user can efficiently create an examination report.

### Other

### Execution of Audio Recognition during Specific Period

The audio recognition unit 62B (processor) can execute the audio recognition using the set audio recognition dictionary during a specific period after the setting (period in which predetermined conditions are satisfied). “Predetermined conditions” may be the output of the recognition results from the image recognizer, may be conditions regarding the output contents, or may specify an execution time itself of the audio recognition (three seconds, five seconds, or the like). In a case of specifying the execution time, it is possible to specify an elapsed time from the dictionary setting, or an elapsed time after the user is notified that the audio input is possible.

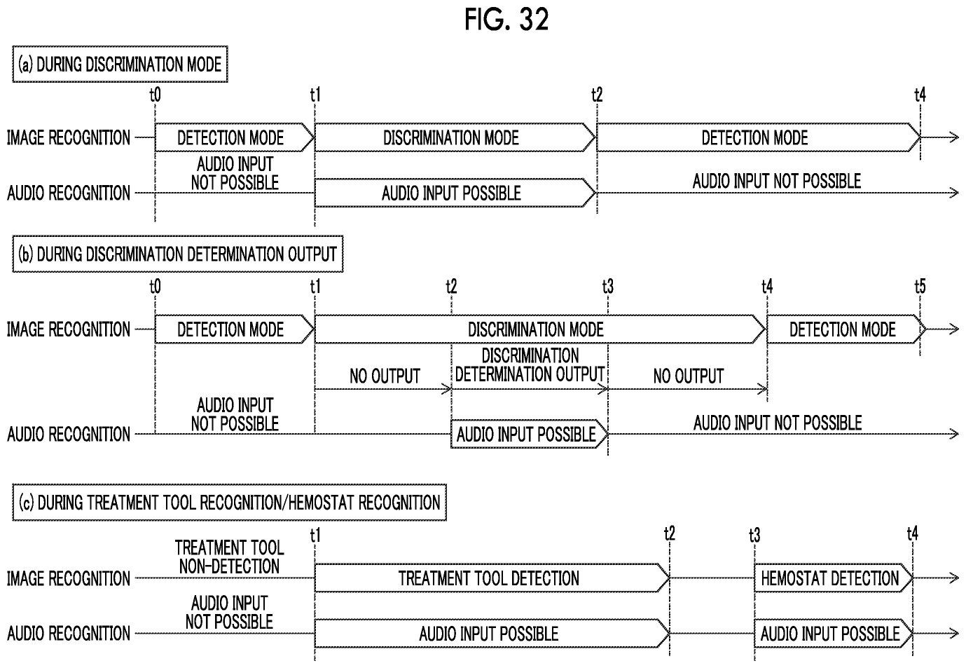

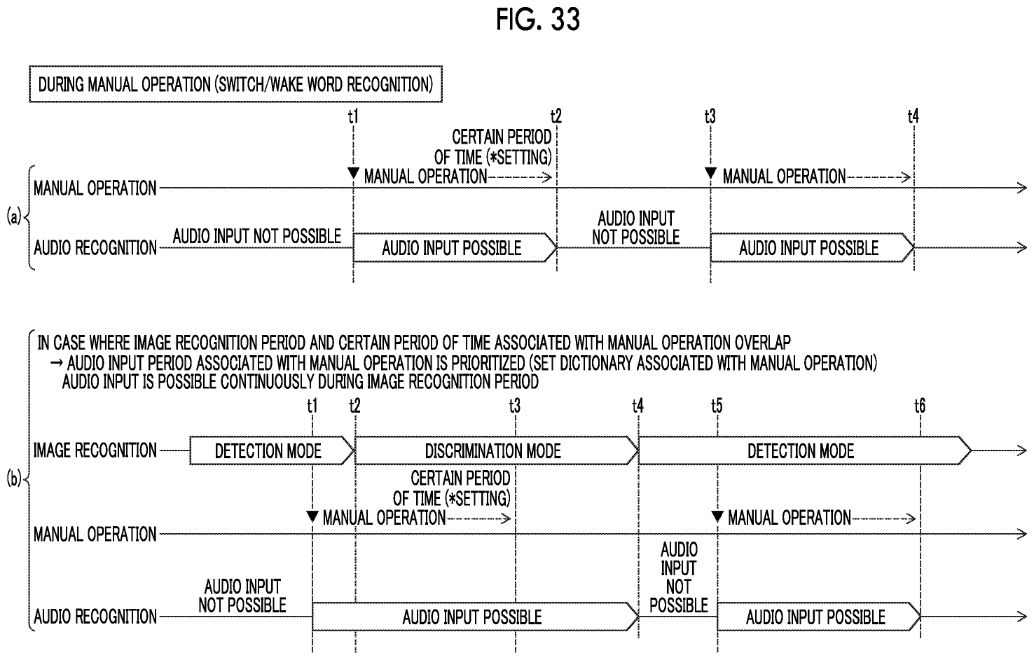

FIG. 32 is a diagram illustrating a state of executing the audio recognition during a specific period. In the example illustrated in the (a) part of FIG. 32, the audio recognition unit 62B performs the audio recognition only during the period in the discrimination mode (period in which the discrimination unit 63B is operating; time point t1 to time point t2). In addition, in the example illustrated in the (b) part of FIG. 32, the audio recognition unit 62B performs the audio recognition only during the period in which the discrimination unit 63B outputs the discrimination result (discrimination determination result) (time point t2 to time point t3). As described above, the discrimination unit 63B can be configured to perform the output in a case where the reliability degree of the discrimination result or the statistical value thereof is equal to or greater than the threshold value. In addition, in the example illustrated in the (c) part of FIG. 32, the audio recognition unit 62B performs the audio recognition only during the period in which the treatment tool detection unit 63D detects the treatment tool (time point t1 to time point t2) and the period in which the hemostat detection unit 63E detects the hemostat (time point t3 to time point t4). Note that, in FIGS. 32 and 33, the acceptance of the audio input trigger and the setting of the audio recognition dictionary are not illustrated.

In this manner, by executing the audio recognition during a specific period, it is possible to reduce the risk of unnecessary recognition or erroneous recognition, and to perform the examination smoothly.

Note that the audio recognition unit 62B may set the period of the audio recognition for each image recognizer, or may set the period of the audio recognition depending on the type of the audio input trigger. In addition, the audio recognition unit 62B may set “predetermined conditions” and “execution time of the audio recognition” on the basis of an instruction input from the user via the input device 50, the operation part 22, or the like. The audio recognition unit 62B and the display control unit 65 can display the result of the audio recognition in the lesion information input box as in the modes described above.

### Audio Recognition after Manual Operation

FIG. 33 is another diagram illustrating a state of executing the audio recognition during a specific period. The (a) part of FIG. 33 illustrates an example of setting the audio recognition dictionary and executing the audio recognition for a certain period of time (time point t1 to time point t2 and time point t3 to time point t4 in the (a) part of FIG. 33) after a manual operation. The audio recognition unit 62B can perform the audio recognition using the user's operation on the input device 50, the operation part 22, or the like as the “manual operation”. Specifically, the “manual operation” may be an operation on various operation devices described above, an input of the wake word via the microphone 51, an operation on the foot switch 52, an imaging instruction of the endoscopic image (video, static image), a switching operation from a detection mode (state in which the lesion part detection unit 63A outputs the result) to a discrimination mode (state in which the discrimination unit 63B outputs the result), or an operation on the operation device connected to the endoscope system 10.

In addition, the (b) part of FIG. 33 illustrates an example of processing in a case where the period of the audio recognition based on the image recognition and the “certain period of time after the manual operation” described above overlap. Specifically, from time point t1 to time point t3, the audio recognition unit 62B prioritizes the audio recognition associated with the manual operation over the audio recognition according to the discrimination result output from the discrimination unit 63B, sets the audio recognition dictionary based on the manual operation, and performs the audio recognition.

In a case where the audio recognition based on the manual operation is prioritized in this manner, the period of the audio recognition based on the image recognition may be continuous with the period of the audio recognition associated with the manual operation. For example, in the example illustrated in the (b) part of FIG. 33, the audio recognition unit 62B sets the audio recognition dictionary based on the discrimination result of the discrimination unit 63B, and performs the audio recognition from time point t3 to time point t4 following the audio recognition period (time point t1 to time point t2) based on the manual operation. Meanwhile, from time point t4 to time point t5, since the audio recognition period based on the manual operation ends, the audio recognition unit 62B does not set the audio recognition dictionary, and does not perform the audio recognition. Similarly, the audio recognition unit 62B sets the audio recognition dictionary based on the manual operation, and performs the audio recognition from time point t5 to time point t6, and after time point t6 at which the audio recognition period ends, the audio recognition unit 62B does not perform the audio recognition.

### Switching Audio Recognition Dictionary According to Quality of Image Recognition

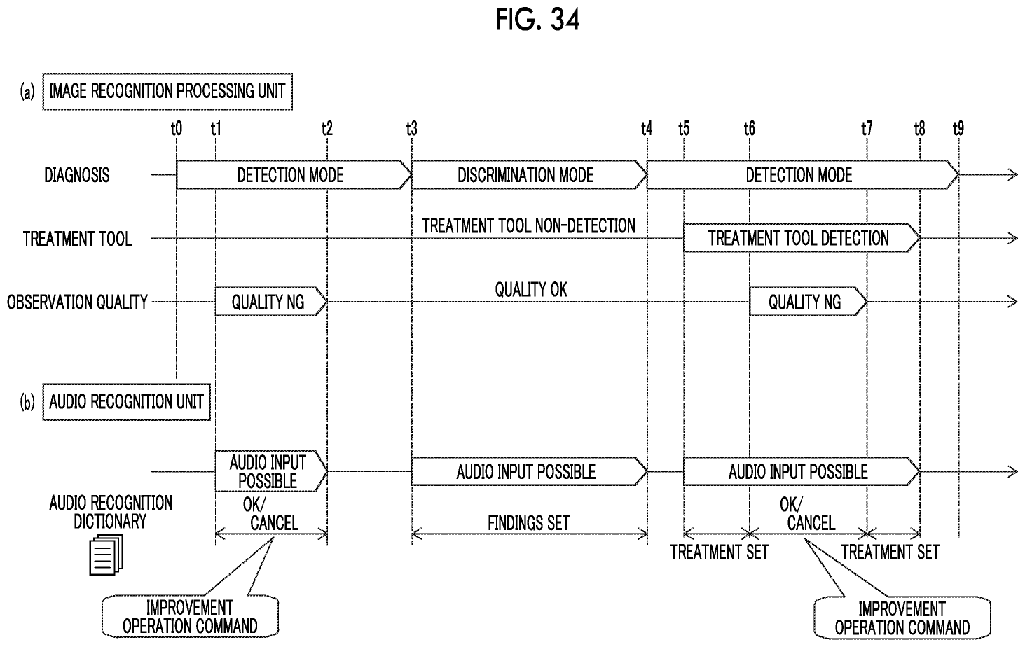

In the audio recognition described above, the audio recognition unit 62B may switch the audio recognition dictionary 62C according to the quality of the image recognition executed by the image recognition processing unit 63, as described below with reference to FIG. 34 (diagram illustrating a state of processing according to the quality of the image recognition).

In a case where the lesion candidate (specific subject) is included in the endoscopic image, the period in which the discrimination unit 63B outputs the discrimination result is the audio recognition period (same as in FIG. 32). In such a situation, as illustrated in the (a) part of FIG. 34, it is assumed that the observation quality (image quality of the endoscopic image) is poor from time point t1 to time point t2 (detection mode; the lesion part detection unit 63A outputs the result). Possible causes of the poor observation quality include, for example, inappropriate exposure or focusing conditions, or obstruction of a visual field due to residue.

In this case, as illustrated in the (b) part of FIG. 34, the audio recognition unit 62B performs the audio recognition from time point t1 to time point t2 at which the audio recognition would not normally be performed (in a case where the image quality is good), and accepts a command for an image quality improvement operation. The audio recognition unit 62B can set an “image quality improvement set” in which, for example, words such as “gas injection, lighting on, sensor sensitivity ‘high’” are registered, as the audio recognition dictionary 62C, and perform the audio recognition.

From time point t3 to time point t4 (discrimination mode: the discrimination unit 63B outputs the result), the audio recognition unit 62B performs the audio recognition using the audio recognition dictionary “findings set” as usual.

In addition, from time point t4 to time point t9, since the mode is the detection mode, the audio recognition unit 62B does not normally perform the audio recognition, and from time point t5 to time point t8, since the treatment tool is detected, the audio recognition unit 62B sets a “treatment set” as the audio recognition dictionary 62C, and performs the audio recognition. However, from time point t6 to time point t7, it is assumed that the observation quality is poor. The audio recognition unit 62B can accept a command for an image quality improvement operation during this period (time point t6 to time point t7) similar to time point t1 to time point t2.

In this manner, in the endoscope system 10, it is possible to flexibly set the audio recognition dictionary according to the observation quality and to perform appropriate audio recognition.

### Application to Endoscope for Upper Digestive Tract

In the embodiments described above, a case has been described in which the present invention is applied to the endoscope system for a lower digestive tract, but the present invention can also be applied to an endoscope for an upper digestive tract.

The embodiments of the present invention have been described above, but the invention is not limited to the above-described aspects and can have various modifications without departing from the scope of the invention.

### EXPLANATION OF REFERENCES

1: endoscopic image diagnosis support system
10: endoscope system
20: endoscope
21: insertion part
21A: distal end portion
21B: bendable portion
21C: soft portion
21a: observation window
21b: illumination window
21c: air/water supply nozzle
21d: forceps outlet
22: operation part
22A: angle knob

What is claimed is:

1. An endoscope system comprising:
an audio input device;
an image sensor that images a subject; and
a processor,
wherein the processor is configured to:
acquire a plurality of medical images obtained by the image sensor imaging the subject in chronological order;
perform a plurality of image recognitions on the acquired plurality of medical images;
set, in a case where a result of any one of the plurality of image recognitions is obtained, an audio recognition dictionary according to the obtained result of any one of the plurality of image recognitions;
display, in a case where the audio recognition dictionary is set, item information indicating an item to be recognized using the audio recognition dictionary, on a display device;
perform audio recognition using the set audio recognition dictionary, in a case where audio input is performed for the item information displayed on the display device; and
display a result of the audio recognition in association with the item information, on the display device.

2. The endoscope system according to claim 1,
wherein the processor performs image recognition for detecting a specific subject, as one of the plurality of image recognitions.

3. The endoscope system according to claim 2,
wherein the specific subject is at least one of a treatment tool and a hemostat.

4. The endoscope system according to claim 3,
wherein, in a case where a result of image recognition indicating detection of the treatment tool is obtained, the processor displays an item of treatment as the item information on the display device,
and/or
in a case where a result of image recognition indicating detection of the hemostat is obtained, the processor displays an item of hemostat, as the item information, on the display device.

5. The endoscope system according to claim 2,
wherein the processor further performs image recognition for discrimination of a lesion detected from the plurality of medical images, as one of the plurality of image recognitions.

6. The endoscope system according to claim 5,
wherein, in a case where a result of the discrimination is obtained, the processor displays an item of diagnosis and findings, as the item information, on the display device.

7. The endoscope system according to claim 1,
wherein, in the audio recognition, the processor recognizes only registered words that are registered in the set audio recognition dictionary, and displays the result of the audio recognition for the registered words on the display device.

8. The endoscope system according to claim 1,
wherein, in the audio recognition, the processor recognizes registered words that are registered in the set audio recognition dictionary and specific words, and displays the result of the audio recognition for the registered words among the recognized words on the display device.

9. The endoscope system according to claim 1,
wherein the processor performs the plurality of image recognitions, using an image recognizer generated by machine learning.

10. The endoscope system according to claim 1,
wherein the processor displays information indicating that the audio recognition dictionary is set, on the display device.

11. The endoscope system according to claim 1,
wherein the processor displays type information indicating a type of the set audio recognition dictionary, on the display device.

12. The endoscope system according to claim 1,
wherein the processor displays the item information and the result of the audio recognition in chronological order.

13. The endoscope system according to claim 1,
wherein the processor displays the item information and the result of the audio recognition on a same display screen as the plurality of medical images.

14. The endoscope system according to claim 1,
wherein the processor
accepts confirmation information indicating confirmation of the audio recognition, and ends, in a case where the confirmation information is accepted, display of the result of the audio recognition and the item information.

15. The endoscope system according to claim 1,
wherein the processor displays the item information and the result of the audio recognition during a display period after the setting, and ends the display in a case where the display period has elapsed.

16. The endoscope system according to claim 15,
wherein the processor displays the item information and the result of the audio recognition during a period in which the audio recognition dictionary is set, as the display period, and ends the display of the item information and the result of the audio recognition in a case where the display period ends.

17. The endoscope system according to claim 15,
wherein the processor displays the item information and the result of the audio recognition during a period with a length according to the obtained result of any one of the plurality of image recognitions, as the display period, and ends the display of the item information and the result of the audio recognition in a case where the display period ends.

18. The endoscope system according to claim 15,
wherein the processor ends the display of the item information and the result of the audio recognition in a case where a state in which a specific subject is recognized in the plurality of medical images ends.

19. The endoscope system according to claim 15,
wherein the processor displays a remaining time of the display period on a screen of the display device.

20. The endoscope system according to claim 1,
wherein the processor
displays a candidate for recognition in the audio recognition on the display device, and
confirms the result of the audio recognition on the basis of a selection operation of a user according to the display of the candidate.

21. The endoscope system according to claim 20,
wherein the processor accepts the selection operation via an operation device different from the audio input device.

22. The endoscope system according to claim 1,
wherein the processor records the plurality of medical images, the item information, and the result of the audio recognition in a recording device such that the plurality of medical images are associated with the item information, and the result of the audio recognition.

23. A medical information processing apparatus comprising
a processor configured to:
acquire a plurality of medical images obtained by an image sensor imaging a subject in chronological order;
perform a plurality of image recognitions on the acquired plurality of medical images;
set, in a case where a result of any one of the plurality of image recognitions is obtained, an audio recognition dictionary according to the obtained result of any one of the plurality of image recognitions;
display, in a case where the audio recognition dictionary is set, item information indicating an item to be recognized using the audio recognition dictionary, on a display device;
perform audio recognition using the set audio recognition dictionary, in a case where audio input is performed for the item information displayed on the display device; and
display a result of the audio recognition in association with the item information, on the display device.

24. A medical information processing method executed by an endoscope system including an audio input device, an image sensor that images a subject, and a processor, the medical information processing method comprising:
acquiring, by the processor, a plurality of medical images obtained by the image sensor imaging the subject in chronological order;
performing, by the processor, a plurality of image recognitions on the acquired plurality of medical images;
setting, by the processor, in a case where a result of any one of the plurality of image recognitions is obtained, an audio recognition dictionary according to the obtained result of any one of the plurality of image recognitions;
displaying, by the processor, in a case where the audio recognition dictionary is set, item information indicating an item to be recognized using the audio recognition dictionary, on a display device;
performing, by the processor, audio recognition using the set audio recognition dictionary, in a case where audio input is performed for the item information displayed on the display device; and
displaying, by the processor, a result of the audio recognition in association with the item information, on the display device.

25. A non-transitory, tangible recording medium which records thereon a computer-readable code of a program for causing a processor of an endoscope system including an audio input device and an image sensor that images a subject, to implement functions comprising:
acquiring a plurality of medical images obtained by the image sensor imaging the subject in chronological order;
performing a plurality of image recognitions on the acquired plurality of medical images;
setting, in a case where a result of any one of the plurality of image recognitions is obtained, an audio recognition dictionary according to the obtained result of any one of the plurality of image recognitions;
displaying, in a case where the audio recognition dictionary is set, item information indicating an item to be recognized using the audio recognition dictionary, on a display device;
performing, audio recognition using the set audio recognition dictionary, in a case where audio input is performed for the item information displayed on the display device; and
displaying, a result of the audio recognition in association with the item information, on the display device.
